(19) United States
(12) Patent Application Publication
(10) Pub. No.: US 2008/0300199 A1
(43) Pub. Date: Dec. 4, 2008

(54) ANTIBACTERIAL 1,4,5-SUBSTITUTED AMINOGLYCOSIDE ANALOGS

(75) Inventors: Martin Linsell, San Mateo, CA (US); Adam Aaron Goldblum, Berkeley, CA (US); James Aggen, Burlingame, CA (US); Heinz Ernst Moser, San Mateo, CA (US); Stephen Hanessian, Beaconsfield (CA); Kandasamy Pachamuthu, Montreal (CA); Ellen Klegraf, Eltville (DE)

Correspondence Address: SEED INTELLECTUAL PROPERTY LAW GROUP PLLC, 701 FIFTH AVE, SUITE 5400, SEATTLE, WA, 98104, US

(73) Assignee: Achaogen Inc., South San Francisco, CA (US)

(21) Appl. No.: 12/100,981

(22) Filed: Apr. 10, 2008

Related U.S. Application Data
(60) Provisional application No. 60/910,909, filed on Apr. 10, 2007.

(51) Int. Cl. A61K 31/7036; C07G 11/00; A61P 31/04

(52) U.S. Cl. 514/38; 536/16.8

(57) ABSTRACT

The present invention is directed to analogs of aminoglycoside compounds as well as their preparation and use as prophylactic or therapeutics against microbial infection.

### CROSS-REFERENCE TO RELATED APPLICATION

This application claims the benefit under 37 U.S.C. § 119(e) of U.S. Provisional Patent Application No. 60/910,909 filed Apr. 10, 2007. This provisional application is incorporated herein by reference in its entirety.

### BACKGROUND OF THE INVENTION

1. Field of the Invention

The present invention is directed to novel aminoglycoside compounds and synthetic methods for their preparation and use as therapeutic or prophylactic agents.

2. Description of the Related Art

A particular interest in modern drug discovery is the development of novel low molecular weight orally-bioavailable drugs that work by binding to RNA. RNA, which serves as a messenger between DNA and proteins, was thought to be an entirely flexible molecule without significant structural complexity. Recent studies have revealed a surprising intricacy in RNA structure. RNA has a structural complexity rivaling proteins, rather than simple motifs like DNA. Genome sequencing reveals both the sequences of the proteins and the mRNAs that encode them. Since proteins are synthesized using an RNA template, such proteins can be inhibited by preventing their production in the first place by interfering with the translation of the mRNA. Since both proteins and the RNAs are potential drug targeting sites, the number of targets revealed from genome sequencing efforts is effectively doubled. These observations unlock a new world of opportunities for the pharmaceutical industry to target RNA with small molecules.

Classical drug discovery has focused on proteins as targets for intervention. Proteins can be extremely difficult to isolate and purify in the appropriate form for use in assays for drug screening. Many proteins require post-translational modifications that occur only in specific cell types under specific conditions. Proteins fold into globular domains with hydrophobic cores and hydrophilic and charged groups on the surface. Multiple subunits frequently form complexes, which may be required for a valid drug screen. Membrane proteins usually need to be embedded in a membrane to retain their proper shape. The smallest practical unit of a protein that can be used in drug screening is a globular domain. The notion of removing a single alpha helix or turn of a beta sheet and using it in a drug screen is not practical, since only the intact protein may have the appropriate 3-dimensional shape for drug binding. Preparation of biologically active proteins for screening is a major limitation in classical high throughput screening. Quite often the limiting reagent in high throughput screening efforts is a biologically active form of a protein which can also be quite expensive.

For screening to discover compounds that bind RNA targets, the classic approaches used for proteins can be superceded with new approaches. All RNAs are essentially equivalent in their solubility, ease of synthesis or use in assays. The physical properties of RNAs are independent of the protein they encode. They may be readily prepared in large quantity through either chemical or enzymatic synthesis and are not extensively modified in vivo. With RNA, the smallest practical unit for drug binding is the functional subdomain. A functional subdomain in RNA is a fragment that, when removed from the larger RNA and studied in isolation, retains its biologically relevant shape and protein or RNA-binding properties. The size and composition of RNA functional subdomains make them accessible by enzymatic or chemical synthesis. The structural biology community has developed significant experience in identification of functional RNA subdomains in order to facilitate structural studies by techniques such as NMR spectroscopy. For example, small analogs of the decoding region of 16S rRNA (the A-site) have been identified as containing only the essential region, and have been shown to bind antibiotics in the same fashion as the intact ribosome.

The binding sites on RNA are hydrophilic and relatively open as compared to proteins. The potential for small molecule recognition based on shape is enhanced by the deformability of RNA. The binding of molecules to specific RNA targets can be determined by global conformation and the distribution of charged, aromatic, and hydrogen bonding groups off of a relatively rigid scaffold. Properly placed positive charges are believed to be important, since long-range electrostatic interactions can be used to steer molecules into a binding pocket with the proper orientation. In structures where nucleobases are exposed, stacking interactions with aromatic functional groups may contribute to the binding interaction. The major groove of RNA provides many sites for specific hydrogen bonding with a ligand. These include the aromatic N7 nitrogen atoms of adenosine and guanosine, the O4 and O6 oxygen atoms of uridine and guanosine, and the amines of adenosine and cytidine. The rich structural and sequence diversity of RNA suggests to us that ligands can be created with high affinity and specificity for their target.

Although our understanding of RNA structure and folding, as well as the modes in which RNA is recognized by other ligands, is far from being comprehensive, significant progress has been made in the last decade (Chow, C. S.; Bogdan, F. M., Chem. Rev., 1997, 97, 1489, Wallis, M. G.; Schroeder, R., Prog. Biophys. Molec. Biol. 1997, 67, 141). Despite the central role RNA plays in the replication of bacteria, drugs that target these pivotal RNA sites of these pathogens are scarce. The increasing problem of bacterial resistance to antibiotics makes the search for novel RNA binders of crucial importance.

Certain small molecules can bind and block essential functions of RNA. Examples of such molecules include the aminoglycoside antibiotics and drugs such as erythromycin which binds to bacterial rRNA and releases peptidyl-tRNA and mRNA. Aminoglycoside antibiotics have long been known to bind RNA. They exert their antibacterial effects by binding to specific target sites in the bacterial ribosome. For the structurally related antibiotics neamine, ribostamycin, neomycin B, and paromomycin, the binding site has been localized to the A-site of the prokaryotic 16S ribosomal decoding region RNA (Moazed, D.; Noller, H. F., Nature, 1987, 327, 389). Binding of aminoglycosides to this RNA target interferes with the fidelity of mRNA translation and results in miscoding and truncation, leading ultimately to bacterial cell death (Alper, P. B.; Hendrix, M.; Sears, P.; Wong, C., J. Am. Chem. Soc., 1998, 120, 1965).

There is a need in the art for new chemical entities that work against bacteria with broad-spectrum activity. Perhaps the biggest challenge in discovering RNA-binding antibacterial drugs is identifying vital structures common to bacteria that can be disabled by small molecule drug binding. A challenge in targeting RNA with small molecules is to develop a chemical strategy which recognizes specific shapes of RNA. There are three sets of data that provide hints on how to do this: natural protein interactions with RNA, natural product antibiotics that bind RNA, and man-made RNAs (aptamers) that bind proteins and other molecules. Each data set, however, provides different insights to the problem.

Several classes of drugs obtained from natural sources have been shown to work by binding to RNA or RNA/protein complexes. These include three different structural classes of antibiotics: thiostreptone, the aminoglycoside family and the macrolide family of antibiotics. These examples provide powerful clues to how small molecules and targets might be selected. Nature has selected RNA targets in the ribosome, one of the most ancient and conserved targets in bacteria. Since antibacterial drugs are desired to be potent and have broad-spectrum activity these ancient processes fundamental to all bacterial life represent attractive targets. The closer we get to ancient conserved functions the more likely we are to find broadly conserved RNA shapes. It is important to also consider the shape of the equivalent structure in humans, since bacteria were unlikely to have considered the therapeutic index of their RNAs while evolving them.

A large number of natural antibiotics exist, these include the aminoglycosides, kirromycin, neomycin, paromomycin, thiostrepton, and many others. They are very potent, bactericidal compounds that bind RNA of the small ribosomal subunit. The bactericidal action is mediated by binding to the bacterial RNA in a fashion that leads to misreading of the genetic code. Misreading of the code during translation of integral membrane proteins is thought to produce abnormal proteins that compromise the barrier properties of the bacterial membrane.

Antibiotics are chemical substances produced by various species of microorganisms (bacteria, fungi, actinomycetes) that suppress the growth of other microorganisms and may eventually destroy them. However, common usage often extends the term antibiotics to include synthetic antibacterial agents, such as the sulfonamides, and quinolines, that are not products of microbes. The number of antibiotics that have been identified now extends into the hundreds, and many of these have been developed to the stage where they are of value in the therapy of infectious diseases. Antibiotics differ markedly in physical, chemical, and pharmacological properties, antibacterial spectra, and mechanisms of action. In recent years, knowledge of molecular mechanisms of bacterial, fungal, and viral replication has greatly facilitated rational development of compounds that can interfere with the life cycles of these microorganisms.

At least 30% of all hospitalized patients now receive one or more courses of therapy with antibiotics, and millions of potentially fatal infections have been cured. At the same time, these pharmaceutical agents have become among the most misused of those available to the practicing physician. One result of widespread use of antimicrobial agents has been the emergence of antibiotic-resistant pathogens, which in turn has created an ever-increasing need for new drugs. Many of these agents have also contributed significantly to the rising costs of medical care.

When the antimicrobial activity of a new agent is first tested, a pattern of sensitivity and resistance is usually defined. Unfortunately, this spectrum of activity can subsequently change to a remarkable degree, because microorganisms have evolved the array of ingenious alterations discussed above that allow them to survive in the presence of antibiotics. The mechanism of drug resistance varies from microorganism to microorganism and from drug to drug.

The development of resistance to antibiotics usually involves a stable genetic change, heritable from generation to generation. Any of the mechanisms that result in alteration of bacterial genetic composition can operate. While mutation is frequently the cause, resistance to antimicrobial agents may be acquired through transfer of genetic material from one bacterium to another by transduction, transformation or conjugation.

For the foregoing reasons, there is a need for new chemical entities that possess antimicrobial activity. Further, in order to accelerate the drug discovery process, new methods for synthesizing aminoglycoside antibiotics are needed to provide an array of compounds that are potentially new drugs for the treatment microbial infections.

### BRIEF SUMMARY OF THE INVENTION

In one embodiment, the present invention provides compounds having the following formula I:

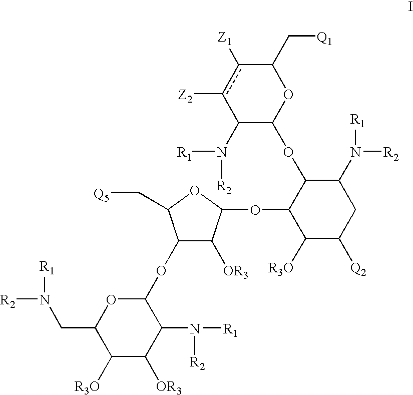

or a stereoisomer, prodrug or pharmaceutically acceptable salt thereof,

wherein:

Q₁ is —OH, a protected hydroxyl, amino or a protected amino group;

Q₂ is

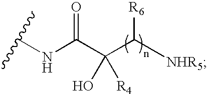

Q₅ is —OH, a protected hydroxyl, amino or a protected amino group;

each R₁ and R₂ is, independently, H or an amino protecting group;

each R₃ is, independently, H or a hydroxyl protecting group;

each R₄, R₅ and R₆ is, independently, H or C₁-C₆ alkyl, or R₄ and R₅ together with the atoms to which they are attached can form a carbocyclic or heterocyclic ring having from 4 to 6 ring atoms, or R₅ and R₆ together with the atoms to which they are attached can form a carbocyclic or heterocyclic ring having from 4 to 6 ring atoms, or R₄ and R₆ together with the atoms to which they are attached can form a carbocyclic ring having from 4 to 6 ring atoms;

n is an integer from 1 to 3; and

each Z₁ and Z₂ is, independently, H, —OH or a protected hydroxyl, and wherein (i) at least one of Z₁ and Z₂ is H, (ii) when Q₁ is —OH or a protected hydroxyl then Z₁ is H, (iii) the two adjacent —CH— groups to which Z₁ and Z₂ are attached may optionally form a double bond, and (iv) when Z₁ and Z₂ are both H and the two adjacent —CH— groups to which Z₁ and Z₂ are attached do not form a double bond, then R₄ and R₅ together with the atoms to which they are attached form a carbocyclic or heterocyclic ring having from 4 to 6 ring atoms, or R₅ and R₆ together with the atoms to which they are attached form a carbocyclic or heterocyclic ring having from 4 to 6 ring atoms, or R₄ and R₆ together with the atoms to which they are attached form a carbocyclic ring having from 4 to 6 ring atoms.

In another embodiment, the present invention provides compounds having the following formula II:

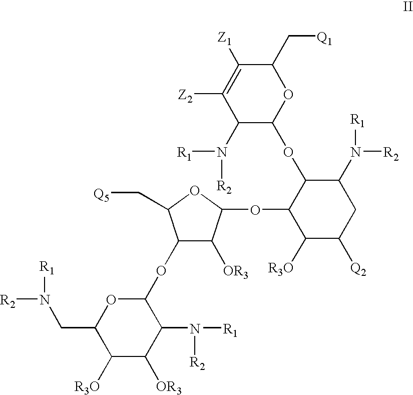

or a stereoisomer, prodrug or pharmaceutically acceptable salt thereof,

wherein:

Q₁ is —OH, a protected hydroxyl, amino or a protected amino group;

Q₂ is

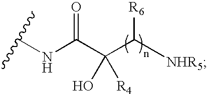

Q₅ is —OH, a protected hydroxyl, amino or a protected amino group;

each R₁ and R₂ is, independently, H or an amino protecting group;

each R₃ is, independently, H or a hydroxyl protecting group;

each R₄, R₅ and R₆ is, independently, H or C₁-C₆ alkyl, or R₄ and R₅ together with the atoms to which they are attached can form a carbocyclic or heterocyclic ring having from 4 to 6 ring atoms, or R₅ and R₆ together with the atoms to which they are attached can form a carbocyclic or heterocyclic ring having from 4 to 6 ring atoms, or R₄ and R₆ together with the atoms to which they are attached can form a carbocyclic ring having from 4 to 6 ring atoms;

n is an integer from 1 to 3; and

each Z₁ and Z₂ is, independently, H, —OH or a protected hydroxyl, and

wherein (i) at least one of Z₁ and Z₂ is H, and (ii) when Q₁ is —OH or a protected hydroxyl then Z₁ is H.

In another embodiment, the present invention provides compounds having the following formula III:

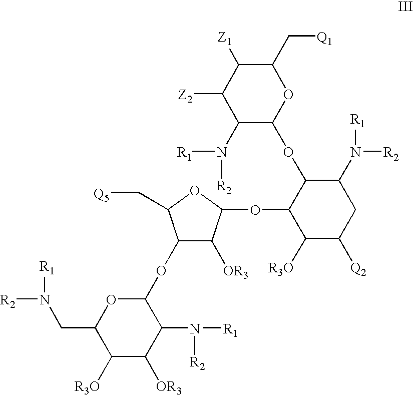

or a stereoisomer, prodrug or pharmaceutically acceptable salt thereof,

wherein:

Q₁ is —OH, a protected hydroxyl, amino or a protected amino group;

Q₂ is

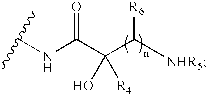

Q₅ is —OH, a protected hydroxyl, amino or a protected amino group;

each R₁ and R₂ is, independently, H or an amino protecting group;

each R₃ is, independently, H or a hydroxyl protecting group;

each R₄, R₅ and R₆ is, independently, H or C₁-C₆ alkyl, or R₄ and R₅ together with the atoms to which they are attached can form a carbocyclic or heterocyclic ring having from 4 to 6 ring atoms, or R₅ and R₆ together with the atoms to which they are attached can form a carbocyclic or heterocyclic ring having from 4 to 6 ring atoms, or R₄ and R₆ together with the atoms to which they are attached can form a carbocyclic ring having from 4 to 6 ring atoms;

n is an integer from 1 to 3; and

each Z₁ and Z₂ is, independently, H, —OH or a protected hydroxyl, and wherein (i) one of Z₁ and Z₂ is H, and (ii) when Q₁ is —OH or a protected hydroxyl then Z₁ is H.

In another embodiment, the present invention provides compounds having the following formula IV:

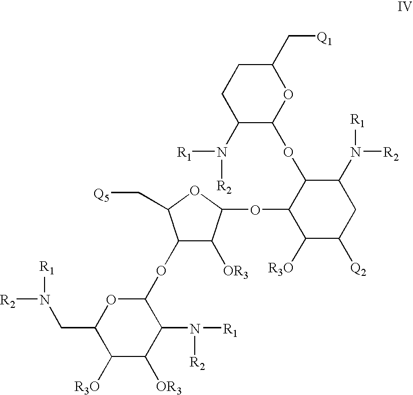

or a stereoisomer, prodrug or pharmaceutically acceptable salt thereof,

wherein:

Q₁ is —OH, a protected hydroxyl, amino or a protected amino group;

Q₂ is

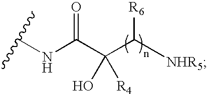

Q₅ is —OH, a protected hydroxyl, amino or a protected amino group;

each R₁ and R₂ is, independently, H or an amino protecting group;

each R₃ is, independently, H or a hydroxyl protecting group;

each R₄, R₅ and R₆ is, independently, H or C₁-C₆ alkyl, and R₄ and R₅ together with the atoms to which they are attached form a carbocyclic or heterocyclic ring having from 4 to 6 ring atoms, or R₅ and R₆ together with the atoms to which they are attached form a carbocyclic or heterocyclic ring having from 4 to 6 ring atoms, or R₄ and R₆ together with the atoms to which they are attached form a carbocyclic ring having from 4 to 6 ring atoms; and

n is an integer from 1 to 3.

In another embodiment, the present invention provides pharmaceutical compositions comprising a compound having formula I, II, III or IV, or a stereoisomer, pharmaceutically acceptable salt or prodrug thereof, and a pharmaceutically acceptable carrier, diluent or excipient.

In another embodiment, the present invention provides methods of using a compound having formula I, II, III or IV in therapy. In particular, the present invention provides a method of treating a bacterial infection in a mammal comprising administering to the mammal an effective amount of a compound having formula I, II, III or IV, or a stereoisomer, prodrug or pharmaceutically acceptable salt thereof.

### DETAILED DESCRIPTION OF THE INVENTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As noted above, in one embodiment, the present invention provides compounds having the following formula I:

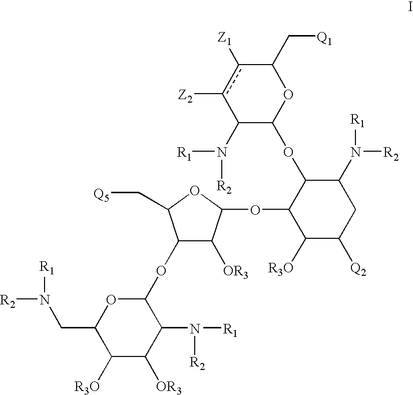

or a stereoisomer, prodrug or pharmaceutically acceptable salt thereof,

wherein:

Q₁ is —OH, a protected hydroxyl, amino or a protected amino group;

Q₂ is

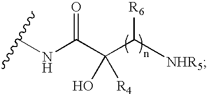

Q₅ is —OH, a protected hydroxyl, amino or a protected amino group;

each R₁ and R₂ is, independently, H or an amino protecting group;

each R₃ is, independently, H or a hydroxyl protecting group;

each R₄, R₅ and R₆ is, independently, H or C₁-C₆ alkyl, or R₄ and R₅ together with the atoms to which they are attached can form a carbocyclic or heterocyclic ring having from 4 to 6 ring atoms, or R₅ and R₆ together with the atoms to which they are attached can form a carbocyclic or heterocyclic ring having from 4 to 6 ring atoms, or R₄ and R₆ together with the atoms to which they are attached can form a carbocyclic ring having from 4 to 6 ring atoms;

n is an integer from 1 to 3; and

each Z₁ and Z₂ is, independently, H, —OH or a protected hydroxyl, and wherein (i) at least one of Z₁ and Z₂ is H, (ii) when Q₁ is —OH or a protected hydroxyl then Z₁ is H, (iii) the two adjacent —CH— groups to which Z₁ and Z₂ are attached may optionally form a double bond, and (iv) when Z₁ and Z₂ are both H and the two adjacent —CH— groups to which Z₁ and Z₂ are attached do not form a double bond, then R₄ and R₅ together with the atoms to which they are attached form a carbocyclic or heterocyclic ring having from 4 to 6 ring atoms, or R₅ and R₆ together with the atoms to which they are attached form a carbocyclic or heterocyclic ring having from 4 to 6 ring atoms, or R₄ and R₆ together with the atoms to which they are attached form a carbocyclic ring having from 4 to 6 ring atoms.

In further embodiments of the compounds of formula I, the two adjacent —CH— groups to which Z₁ and Z₂ are attached form a double bond, and the compounds have the above noted formula II.

In further embodiments of the foregoing, each R₁, R₂ and R₃ are H.

In further embodiments of the foregoing, Q₅ is amino.

In more specific embodiments of the foregoing, Q₁ is amino. In yet further, more specific embodiments of the foregoing, Q₂ is:

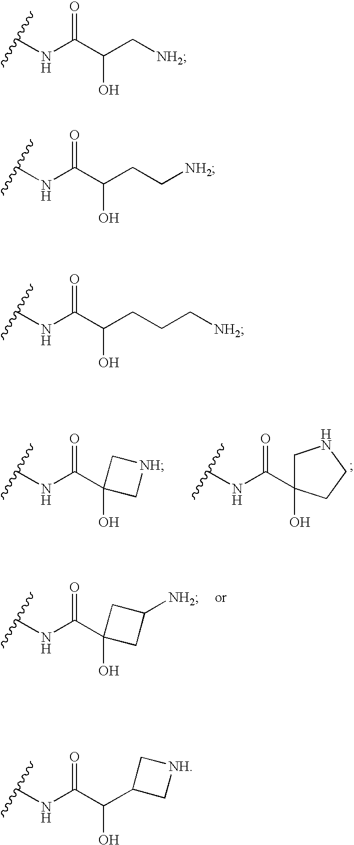

In yet further, more specific embodiments, Z₁ and Z₂ are H, Z₁ is H and Z₂ is —OH, or Z₁ is —OH and Z₂ is H.

In other more specific embodiments of the foregoing, Q₁ is —OH. In yet further, more specific embodiments of the foregoing, Q₂ is:

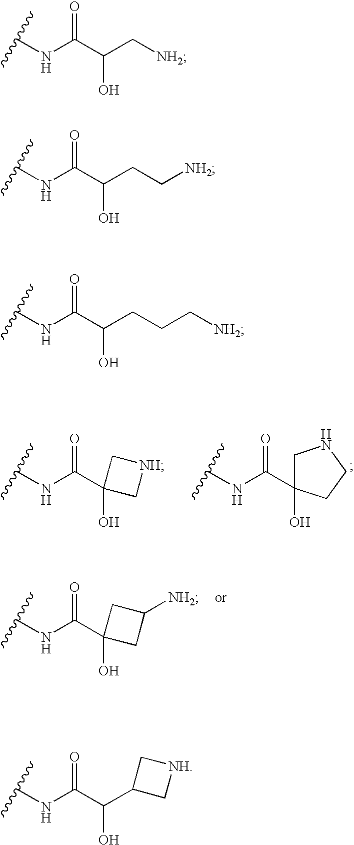

In yet further, more specific embodiments, Z₁ and Z₂ are H, or Z₁ is H and Z₂ is —OH.

In other further embodiments of the foregoing, Q₅ is —OH.

In more specific embodiments of the foregoing, Q₁ is amino. In yet further, more specific embodiments of the foregoing, Q₂ is:

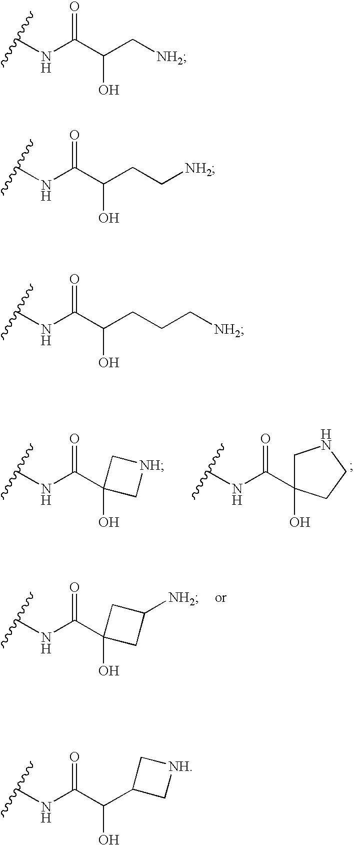

In yet further, more specific embodiments, Z₁ and Z₂ are H, Z₁ is H and Z₂ is —OH, or Z₁ is —OH and Z₂ is H.

In other more specific embodiments of the foregoing, Q₁ is —OH. In yet further, more specific embodiments of the foregoing, Q₂ is:

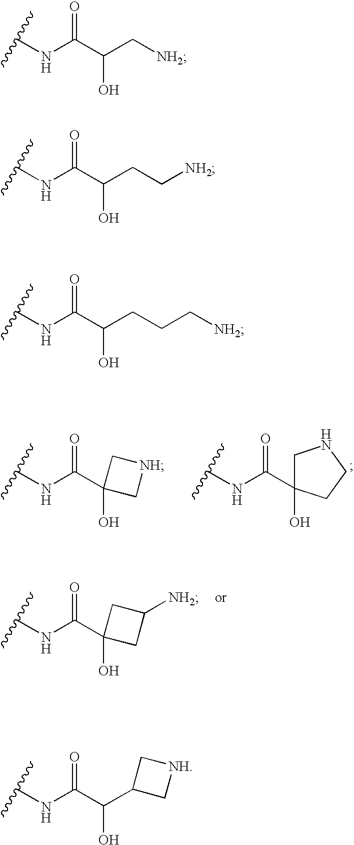

In yet further, more specific embodiments, Z₁ and Z₂ are H, or Z₁ is H and Z₂ is —OH.

In other further embodiments of the compounds of formula I, the two adjacent —CH— groups to which Z₁ and Z₂ are attached do not form a double bond.

In further embodiments of the foregoing, one of Z₁ and Z₂ is H, and the compounds have the above noted formula III.

In further embodiments of the foregoing, each R₁, R₂ and R₃ are H.

In further embodiments of the foregoing, Q₅ is amino.

In more specific embodiments of the foregoing, Q₁ is amino. In yet further, more specific embodiments of the foregoing, Q₂ is:

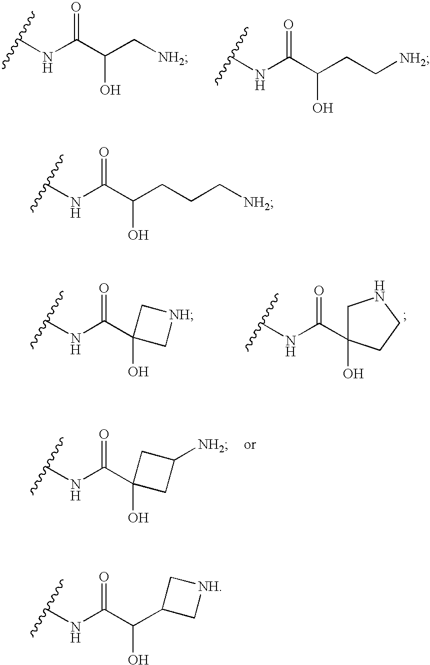

In yet further, more specific embodiments, Z₁ is H and Z₂ is —OH, or Z₁ is —OH and Z₂ is H.

In other more specific embodiments of the foregoing, Q₁ is —OH. In yet further, more specific embodiments of the foregoing, Q₂ is:

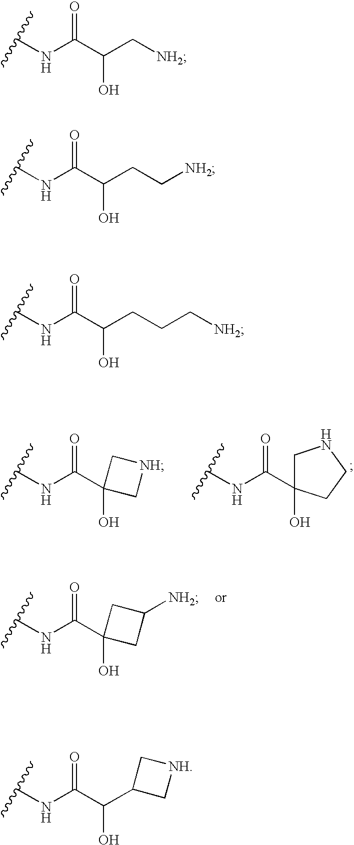

In yet further, more specific embodiments, Z₁ is H and Z₂ is —OH.

In other further embodiments of the foregoing, Q₅ is —OH.

In more specific embodiments of the foregoing, Q₁ is amino. In yet further, more specific embodiments of the foregoing, Q₂ is:

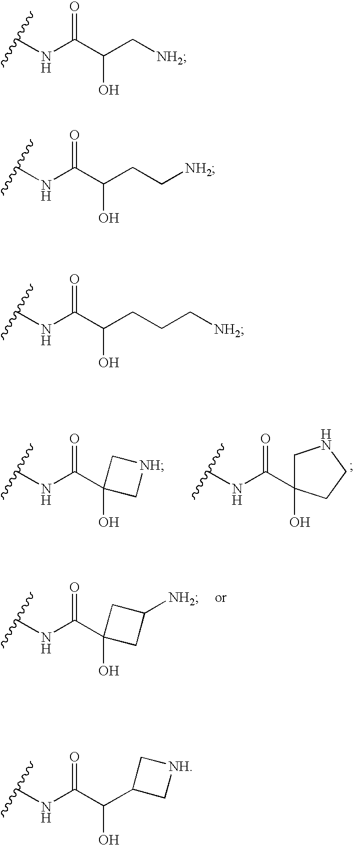

In yet further, more specific embodiments, Z₁ is H and Z₂ is —OH, or Z₁ is —OH and Z₂ is H.

In other more specific embodiments of the foregoing, Q₁ is —OH. In yet further, more specific embodiments of the foregoing, Q₂ is:

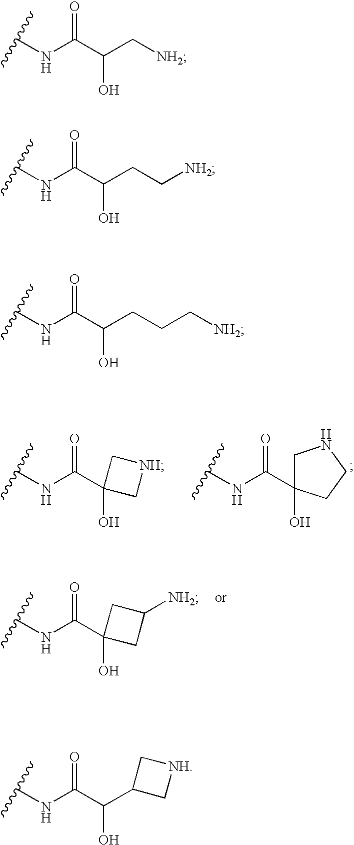

In yet further, more specific embodiments, Z₁ is H and Z₂ is —OH.

In other further embodiments of the foregoing, Z₁ and Z₂ are both H, and the compounds have the above noted formula IV.

In further embodiments of the foregoing, each R₁, R₂ and R₃ are H.

In further embodiments of the foregoing, Q₅ is amino.

In more specific embodiments of the foregoing, Q₁ is amino. In yet further, more specific embodiments of the foregoing, Q₂ is:

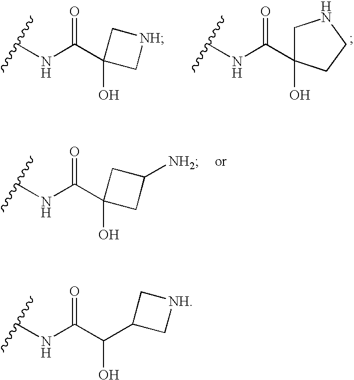

In other more specific embodiments of the foregoing, Q₁ is —OH. In yet further, more specific embodiments of the foregoing, Q₂ is:

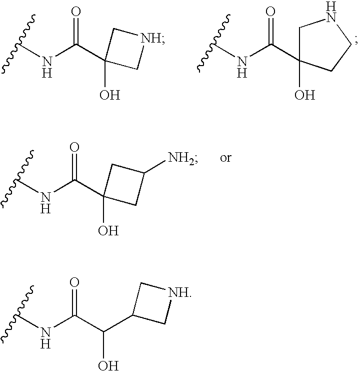

In other further embodiments of the foregoing, Q₅ is —OH.

In more specific embodiments of the foregoing, Q₁ is amino. In yet further, more specific embodiments of the foregoing, Q₂ is:

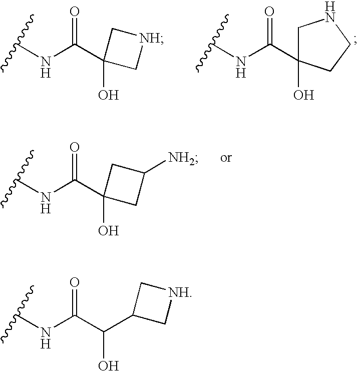

In other more specific embodiments of the foregoing, Q₁ is —OH. In yet further, more specific embodiments of the foregoing, Q₂ is:

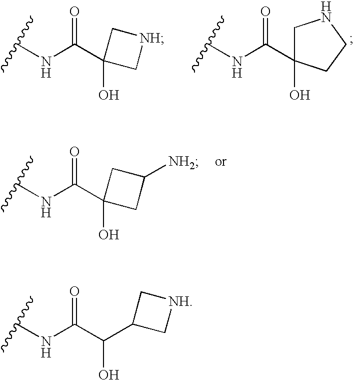

In other further embodiments, the foregoing compounds of formula I have the following configuration:

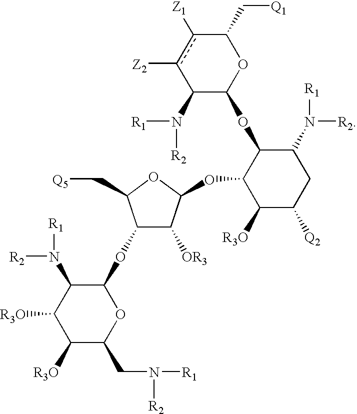

In other further embodiments, the foregoing compounds of formula II have the following configuration:

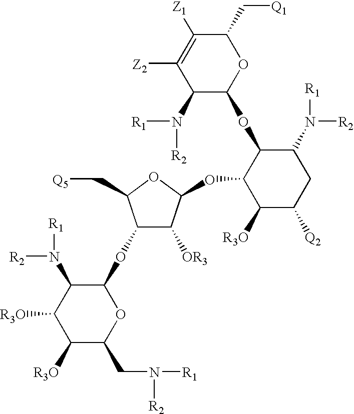

In other further embodiments, the foregoing compounds of formula III have the following configuration:

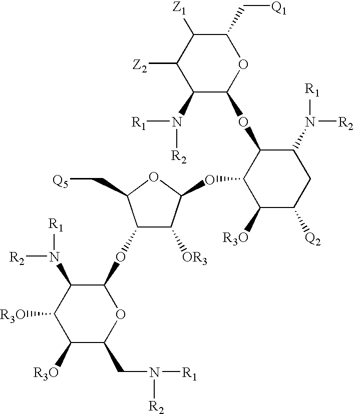

In other further embodiments, the foregoing compounds of formula IV have the following configuration:

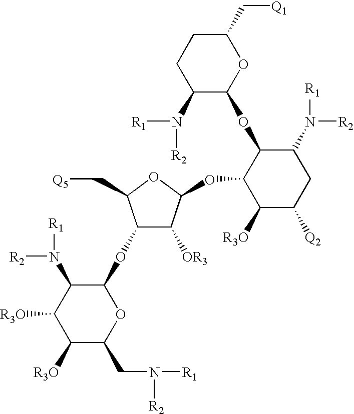

In another embodiment, the present invention provides aminoglycoside compounds having the following formula V:

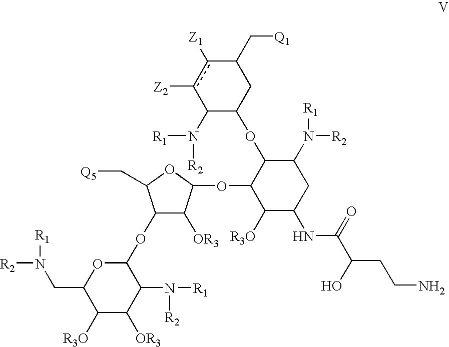

or a stereoisomer, prodrug or pharmaceutically acceptable salt thereof,

wherein:

Q₁ is —OH, a protected hydroxyl, amino or a protected amino group;

Q₅ is —OH, a protected hydroxyl, amino or a protected amino group;

each R₁ and R₂ is, independently, H or an amino protecting group;

each R₃ is, independently, H or a hydroxyl protecting group; and

each Z₁ and Z₂ is, independently, H, —OH or a protected hydroxyl, and wherein (i) at least one of Z₁ and Z₂ is H, (ii) when Q₁ is —OH or a protected hydroxyl then Z₁ is H, (iii) the two adjacent —CH— groups to which Z₁ and Z₂ are attached may optionally form a double bond, and (iv) when Z₁ and Z₂ are both H and the two adjacent —CH— groups to which Z₁ and Z₂ are attached do not form a double bond, then R₄ and R₅ together with the atoms to which they are attached form a carbocyclic or heterocyclic ring having from 4 to 6 ring atoms, or R₅ and R₆ together with the atoms to which they are attached form a carbocyclic or heterocyclic ring having from 4 to 6 ring atoms, or R₄ and R₆ together with the atoms to which they are attached form a carbocyclic ring having from 4 to 6 ring atoms.

In further embodiments of the compounds having the following formula I:

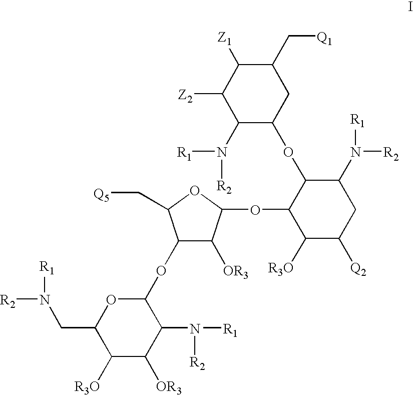

wherein each R₁, R₂ and R₃ are H.

In further embodiments of the foregoing, Q₅ is amino. In more specific embodiments, Q₁ may be amino or —OH. In yet further, more specific, embodiments of the foregoing, Q₂ is:

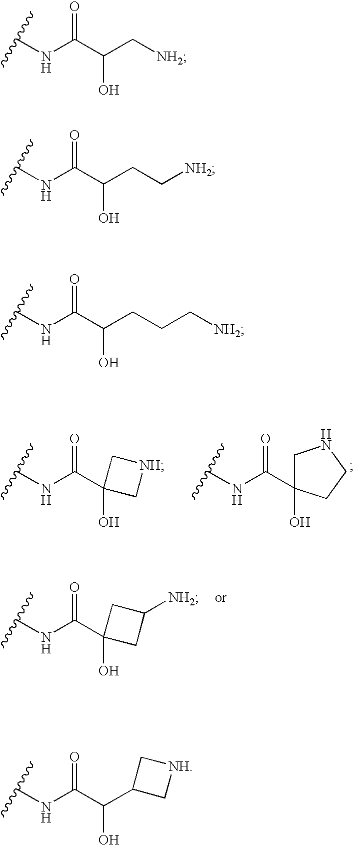

In yet further, more specific, embodiments of the foregoing, Z₁ and Z₂ are H, Z₁ is H and Z₂ is —OH, or Z₁ is —OH and Z₂ is H (provided that when Q₁ is —OH, then Z₁ is H). In yet further, more specific, embodiments of the foregoing, the two adjacent —CH— groups to which Z₁ and Z₂ are attached form a double bond.

In other further embodiments of the foregoing, Q₅ is —OH. In more specific embodiments, Q₁ may be amino or —OH. In yet further, more specific, embodiments of the foregoing, Q₂ is:

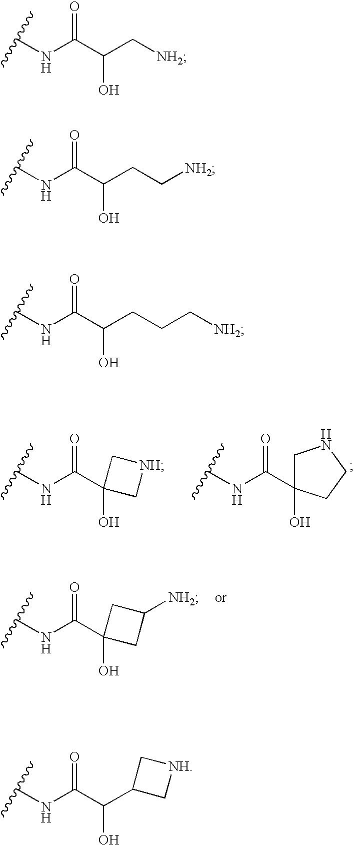

In yet further, more specific, embodiments of the foregoing, Z₁ and Z₂ are H, Z₁ is H and Z₂ is —OH, or Z₁ is —OH and Z₂ is H (provided that when Q₁ is —OH, then Z₁ is H). In yet further, more specific, embodiments of the foregoing, the two adjacent —CH— groups to which Z₁ and Z₂ are attached form a double bond.

It is understood that any embodiment of the compounds of formula I, II, III, IV or V, as set forth above, and any specific substituent set forth herein for a substituent group in the compounds of formula I, II, III, IV or V, as set forth above, may be independently combined with other embodiments and/or substituents of compounds of formula I, II, III, IV or V to form embodiments of the inventions not specifically set forth above. In addition, in the event that a list of substitutents is listed for any particular substituent group in a particular embodiment and/or claim, it is understood that each individual substituent may be deleted from the particular embodiment and/or claim and that the remaining list of substituents will be considered to be within the scope of the invention.

The term “alkyl,” as used herein, refers to a saturated straight or branched hydrocarbon radical containing up to twenty four carbon atoms. Examples of alkyl groups include, but are not limited to, methyl, ethyl, propyl, butyl, isopropyl, n-hexyl, octyl, decyl, dodecyl and the like. Alkyl groups containing from 1 to 6 carbon atoms are referred to as C₁-C₆ alkyl.

The terms “carbocycle” or “carbocyclic ring,” as used herein, refers to a non-aromatic monocyclic or polycyclic hydrocarbon radical consisting solely of carbon and hydrogen atoms, which is saturated or unsaturated. Monocyclic radicals include, for example, cyclopropyl, cyclobutyl, cyclopentyl, cyclohexyl, cycloheptly, cyclooctyl, and the like. Polycyclic radicals include, for example, adamantine, norbornane, decalinyl, 7,7-dimethyl-bicyclo[2.2.1]heptanyl, and the like.

The terms “heterocycle” or “heterocyclic ring,” as used herein, refers to a non-aromatic monocyclic or polycyclic radical that includes at least one heteroatom selected from the group consisting of nitrogen, oxygen and sulfur. The heterocycle or heterocyclic ring may be a monocyclic, bicyclic, tricyclic or tetracyclic ring system, which may include fused or bridged ring systems; and the nitrogen, carbon or sulfur atoms in the heterocycle or heterocyclic ring may be optionally oxidized; the nitrogen atom may be optionally quaternized; and the heterocycle or heterocyclic ring may be partially or fully saturated. Heterocycles and heterocyclic ring include, for example, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, and the like.

The term “protecting group,” as used herein, refers to a labile chemical moiety which is known in the art to protect reactive groups including without limitation, hydroxyl and amino groups, against undesired reactions during synthetic procedures. Hydroxyl and amino groups which protected with a protecting group are referred to herein as “protected hydroxyl groups” and “protected amino groups”, respectively. Protecting groups are typically used selectively and/or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999).

Groups can be selectively incorporated into aminoglycosides of the invention as precursors. For example an amino group can be placed into a compound of the invention as an azido group that can be chemically converted to the amino group at a desired point in the synthesis. Generally, groups are protected or present as a precursor that will be inert to reactions that modify other areas of the parent molecule for conversion into their final groups at an appropriate time. Further representative protecting or precursor groups are discussed in Agrawal, et al., Protocols for Oligonucleotide Conjugates, Eds, Humana Press; New Jersey, 1994; Vol. 26 pp. 1-72.

Examples of “hydroxyl protecting groups” include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Examples of “amino protecting groups” include, but are not limited to, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide-protecting groups, such as 2-nitrobenzenesulfonyl; and imine and cyclic imide protecting groups, such as phthalimido and dithiasuccinoyl.

In one aspect of the present invention aminoglycoside compounds having formula I are modified by covalent attachment of one or more conjugate groups that modify one or more properties of the compounds, including but not limited to pharmakodynamic, pharmacokinetic, binding, absorption, cellular distribution, cellular uptake, charge and clearance. Conjugate groups are routinely used in the chemical arts with a preferred list including, without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Reporter groups that are suitable as conjugate groups include any moiety that can be detected by, for example, spectroscopic means. Examples of reporter groups include dyes, fluorophores, phosphors, radiolabels, and the like. In some embodiments, the reporter group is biotin, flourescein, rhodamine, coumarin, or related compounds. Reporter groups can also be attached to other conjugate moieties. Conjugate moieties can be attached directly to a compound of the present invention or through a linker group or bifunctional linking moiety (linker or tether).

Aminoglycoside compounds of the present invention may be prepared according to established organic synthetic methods. In a particular method, as set forth in the Examples below, paromomycin (or paromomycin salt, which is commercially available from various sources, including Sigma-Aldrich Co.) is selected protected such that the I position can be selectively functionalized.

The synthesized aminoglycoside compounds of the present invention can be separated from reaction mixtures and further purified by methods including but not limited to column chromatography, high pressure liquid chromatography and recrystallization. Further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)— or (S)—, α or β, or as (D)- or (L)- such as for amino acids et al. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

It has been found that the compounds of the present invention possess antibacterial activity against a wide spectrum of gram positive and gram negative bacteria, as well as enterobacteria and anaerobes. The compounds, by reason of their in vitro activity, may be used in scrub solutions for surface inhibition of bacterial growth, e.g., in sterilization of glasswear or as an additive in fabric laundering compositions. Representative susceptible organisms generally include those gram positive and gram negative, aerobic and anaerobic organisms whose growth can be inhibited by the compounds of the invention such as Staphylococcus, Lactobacillus, Streptococcus, Sarcina, Escherichia, Enterobacter, Klebsiella, Pseudomonas, Acinetobacter, Proteus, Campylobacter, Citrobacter, Nisseria, Baccillus, Bacteroides, Peptococcus, Clostridium, Salmonella, Shigella, Serratia, Haemophilus, Brucella and other organisms.

In addition, as described in Example 21 herein, surprisingly improved activity on certain strains of aminoglycoside-resistant Pseudomonas aeruginosa, particularly those strains expressing efflux-based resistance alone or in combination with aminoglycoside modifying enzymes (AMEs), has been associated with compounds having formula II (particularly, those in which Z₁ and Z₂ are both hydrogen).

Accordingly there is provided a method of treating bacterial infection in a mammal comprising administering to the mammal, for example a human, an effective amount of a compound of the invention. By “effective amount” is meant an amount of compound which upon administration is capable of reducing or preventing proliferation of the bacteria or reducing or preventing symptoms associated with the bacterial infection. The actual amount of compound administered and the route of administration will depend upon the particular disease or bacteria as well as other factors such as the size, age, sex and ethnic origin of the individual being treated and is determined by routine analysis. The compounds of the invention may also be formulated into compositions together with pharmaceutically acceptable carriers for parenteral injection, for oral administration in solid or liquid form, for rectal administration, and the like. In methods of the invention, the compound may be administered orally (including buccal, sublingual, inhalation), nasally, rectally, vaginally, intravenously, intradermally, subcutaneously and topically. Compounds will be formulated into compositions suitable for administration for example with suitable carriers, diluents, thickeners, adjuvants, etc., as are routine in the formulation art. Compositions of the invention may also include additional active ingredients. Dosage forms include solutions, powders, tables, capsules, gel capsules, suppositories, topical ointments and creams and aerosols for inhalation.

Formulations for non-parenteral administration may include sterile aqueous solutions which may also contain buffers, diluents and other suitable additives. Pharmaceutically acceptable organic or inorganic carrier substances suitable for non-parenteral administration which do not deleteriously react with compounds of the invention can be used. Suitable pharmaceutically acceptable carries include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like. The formulations can be sterilized and, if desired, mixed with auxiliary agents, e.g., lubricants, preservatives, stabilizers, wetting agents, emulsifiers, salts for influencing osmotic pressure, buffers, colorings flavorings and/or aromatic substances and the like which do not deleteriously react with compounds of the invention. Aqueous suspensions may contain substances which increase the viscosity of the suspension including, for example, sodium carboxymethylcellulose, sorbitol and/or dextran. Optionally, the suspension may also contain stabilizers.

In a preferred embodiment, compounds of the invention are administered via oral delivery. Compositions for oral administration include powders or granules, suspensions or solutions in water or non-aqueous media, capsules, sachets, troches, tablets or SECs (soft elastic capsules or caplets). Thickeners, flavoring agents, diluents, emulsifiers, dispersing aids, carrier substances of binders may be desirably added to such formulations. The use of such formulations has the effect of delivering the nucleic acid to the alimentary canal for exposure to the mucosa thereof. Accordingly, the formulation can consist of material effective in protecting the compound from pH extremes of the stomach, or in releasing the compound over time, to optimize the delivery thereof to a particular mucosal site. Enteric coatings for acid-resistant tablets, capsules and caplets are known in the art and typically include acetate phthalate, propylene glycol and sorbitan monoleate.

Various methods for producing formulations for alimentary delivery are well known in the art. See, generally, Naim, Chapter 83; Block, Chapter 87; Rudnic et. al., Chapter 89; and Longer et. al., Chapter 91 In: Remington's Pharmaceutical Sciences, 18^{th} Ed., Gennaro, ed., Mack Publishing Co., Easton, Pa., 1990. The formulations of the invention can be converted in a known manner into the customary formulations, such as tablets, coated tablets, pills, granules, aerosols, syrups, emulsions, suspensions and solutions, using inert, non-toxic, pharmaceutically suitable excipients or solvents. The therapeutically active compound should in each case be present in a concentration of about 0.5% to about 95% by weight of the total mixture, that is to say in amounts which are sufficient to achieve the desired dosage range. The formulations are prepared, for example, by extending the active compounds with solvents and/or excipients, if appropriate using emulsifying agents and/or dispersing agents, and, for example, in the case where water is used as the diluent, organic solvents can be used as auxiliary solvents if appropriate.

Compositions may be formulated in a conventional manner using additional pharmaceutically acceptable carriers or excipients as appropriate. Thus, the composition may be prepared by conventional means with additional carriers or excipients such as binding agents (e.g., pregelatinised maize starch, polyvinylpyrrolidone or hydroxypropyl methylcellulose); filters (e.g., lactose, microcrystalline cellulose or calcium hydrogen phosphate); lubricants (e.g., magnesium stearate, talc or silica); disintegrates (e.g., starch or sodium starch glycolate); or wetting agents (e.g., sodium lauryl sulfate). Tablets may be coated by methods will known in the art. The preparations may be also contain flavoring, coloring and/or sweetening agents as appropriate.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided soled carriers or both, and then, if necessary, shaping the product.

Formulations of the present invention suitable for oral administration may be presented as discrete units such as capsules, cachets or tables each containing predetermined amounts of the active ingredients; as powders or granules; as solutions or suspensions in an aqueous liquid or a non-aqueous liquid; or as oil-in-water emulsions or water-in-oil liquid emulsions. A tablet may be made by compression or molding, optionally with one or more accessory ingredients. Compressed tablets may be prepared by compressing in a suitable machine, the active ingredients in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredients therein.

Included within the scope of the present invention are the pharmaceutically acceptable salts of the foregoing compounds. As used herein, the term “pharmaceutically acceptable salts” refers to non-toxic acid addition salts and alkaline earth metal salts of the compounds of the invention. The salts can be prepared in situ during the final isolation and purification of the compounds of the invention, or separately by reacting the free base or acid functions with a suitable organic acid or base. Representative acid addition salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, glucoheptonate, lactobionate, lauryl sulfate salts and the like. Representative alkali or alkaline earth metal salts include the sodium, calcium, potassium and magnesium salts.

Included within the scope of the present invention are prodrugs of the foregoing compounds. As used herein, the term “prodrug” refers to a compound that may be converted under physiological conditions or by solvolysis to a biologically active compound of the present invention. Thus, the term “prodrug” refers to a metabolic precursor of a compound of the present invention that is pharmaceutically acceptable. A prodrug may be inactive when administered to a subject in need thereof, but is converted in vivo to an active compound. Prodrugs are typically rapidly transformed in vivo to yield the active compound, for example, by hydrolysis in blood. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam)). A discussion of prodrugs is also provided in Higuchi, T., et al., “Pro-drugs as Novel Delivery Systems,” A.C.S. Symposium Series, Vol. 14, and in Bioreversible Carriers in Drug Design, Ed. Edward B. Roche, American Pharmaceutical Association and Pergamon Press, 1987, both of which are incorporated in full by reference herein.

The term “prodrug” is also meant to include any covalently bonded carriers, which release an active compound of the present invention in vivo when such prodrug is administered to a mammalian subject. Prodrugs are generally prepared by modifying functional groups in a way such that the modification is cleaved, either by routine manipulation or in vivo, yielding the parent compound. Prodrugs include, for example, compounds of the present invention wherein hydroxy, amine or sulfhydryl groups are bonded to any group that, when administered to a mammalian subject, cleaves to form the hydroxy, amine or sulfhydryl groups. Thus, representative examples of prodrugs include (but are not limited to) acetate, formate and benzoate derivatives of alcohol and amine functional groups of the compounds of the present invention. Further, in the case of a carboxylic acid (—COOH), esters may be employed, such as methyl esters, ethyl esters, and the like.

The invention disclosed herein is also meant to encompass the in vivo metabolic products of the disclosed compounds. Such products may result from, for example, the oxidation, reducation, hydrolysis, amidation, esterification, and the like of the administered compound, primarily due to enzymatic processes. Accordingly, the invention includes compounds produced by a process comprising contacting a compound of this invention with a mammal for a period of time sufficient to yield a metabolic product thereof. Such products are typically are identified by administering a radiolabelled compound of the invention in a detectable dose to an animal, such as rat, mouse, guinea pig, monkey, or to human, allowing sufficient time for metabolism to occur, and isolating its coversion products from the urine, blood or other biological samples.

The following examples are provided for purposes of illustration, not limitation.

### EXAMPLES

### Example 1

### Synthesis of Compound 2 (4′,6′-O-benzylidene-penta-N-benzyloxycarbonyl paromomycin)

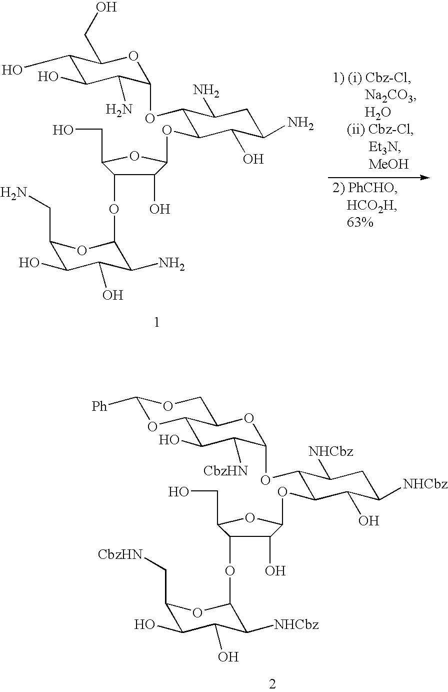

Sodium carbonate (55.0 g, 0.523 mol) and Cbz-Cl (20.00 mL, 0.139 mol) were added to paromomycin sulfate (30.00 g, 0.0271 mol) in water (500 mL). After 35 hours under vigorous stirring, the water was decanted and the white precipitate was washed with water twice. A solution of triethylamine (97.00 mL, 0.697 mol) in methanol (600 mL) was added, followed by Cbz-Cl (25.00 mL, 0.174 mol). After 24 hours, dimethylamine (100 mL of a 40% aqueous solution) was added to quench the remaining Cbz-Cl. The solvents were evaporated and the oil was washed with 3% methanol in ether twice and water. The resulting sticky solid was co-distilled with pyridine (200 mL) three times and at ½ of the volume of the third co-distillation, toluene (200 mL) was added and the solvents were evaporated to dryness. Another co-distillation with toluene (300 mL) was done before heating the flask at 60° C. under 10 mm Hg vacuum for 12 hours. Freshly distilled benzaldehyde (400 mL) was added to the resulting white solid and sonication was used to form a solution. To the stirred mixture was added 4 angstrom molecular sieves (15 g) and formic acid (20.00 mL, 0.530 mol). After stirring for 12 hours at room temperature, the mixture was added dropwise to a stirred ice-cold solution of saturated aqueous Na₂CO₃, extracted with ethyl acetate (3 times), and the organic layer was washed with water, brine and dried over Na₂SO₄. The solvent was evaporated to dryness and excess benzaldehyde was removed under vacuum to afford a crude solid, which was purified by flash column chromatography over silica gel (3% MeOH/CH₂Cl₂) to obtain pure Compound 2 (23.89 g, 63%).

The spectroscopic analysis of the resulting material was consistent with data reported in the literature for the identical material (Hanessian S., Takamoto T., Massé R., Patil G.; Aminoglycoside antibiotics: Chemical conversion of neomycin B, paromomycin, and lividomycin B into bioactive pseudosaccharides; Can. J. Chem., 1978, 56, 1482).

### Example 2

### Synthesis of Compound 3

### Synthesis of Compound 3a

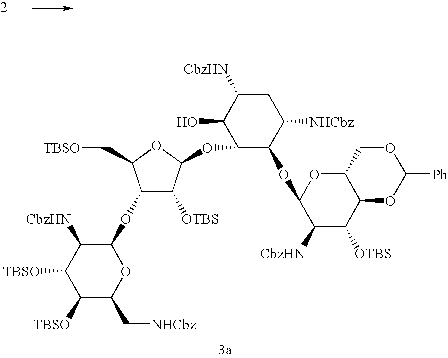

To a stirred solution of Compound 2 (1.35 g, 0.98 mmol) in dry dichloromethane (20 mL) was added 2,4,6-collidine (1.07 g, 8.82 mmol) and TBSOTf (1.811 g, 6.86 mmol) at 0° C. The reaction mixture was slowly brought to room temperature and stirred for 12 hours. A few drops of water was added to quench the excess TBSOTf, followed by extraction with dichloromethane. The organic layer was washed with brine and dried over anhydrous Na₂SO₄, followed by concentration of the solvent to give the corresponding crude product. The crude product was purified by flash column chromatography to give Compound 3a (1.048 g, 55%).

[α]_{D}=+16° (c 0.6, CHCl₃). ESI/MS calcd for C₁₀₀H₁₄₉N₅O₂₄Si₅ (M+H⁺) 1944.94; found 1946.

### Synthesis of Compound 3b

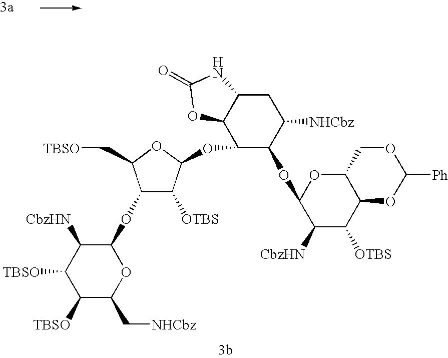

To a stirred solution of Compound 3a (330 mg, 0.17 mmol) in dry DMF (6 mL) was added 60% NaH in mineral oil (8 mg) at 0° C. with stirring continued for an additional 6 hours at 0° C. A few drops of saturated ammonium chloride solution were added, followed by extraction with ethyl acetate. The organic layer was washed with brine and dried over anhydrous Na₂SO₄, followed by concentration of the solvent yielding the corresponding crude product. The crude product was purified by flash column chromatography to yield Compound 3b (180 mg, 58%).

[α]_{D}=+18° (c 0.5, CHCl₃). ESI/MS calcd for C₉₃H₁₄₁N₅O₂₃Si₅ (M+H⁺) 1836.89; found 1837.6.

### Synthesis of Compound 3c

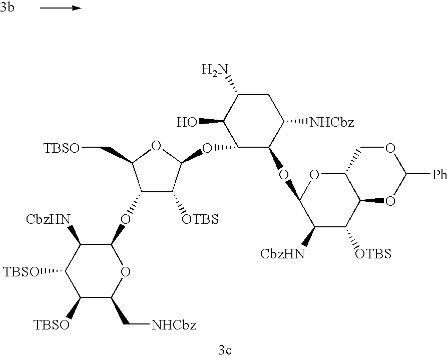

To a stirred solution of Compound 3b (190 mg, 0.1 mmol) in DMF (7 mL) was added 0.7 mL of aqueous LiOH (9 mg, 0.21 mmol) with stirring continued for an additional 3 hours at room temperature. A few drops of saturated ammonium chloride solution was added, followed by extraction with ethyl acetate. The organic layer was washed with brine and dried over anhydrous Na₂SO₄, followed by concentration of the solvent yielding the corresponding crude product. The crude product was purified by flash column chromatography to yield Compound 3 c (100 mg, 53%).

[α]_{D}=+13° (c 0.3, CHCl₃). ESI/MS calcd for C₉₂H₁₄₃N₅O₂₂Si₅ (M+H⁺) 1810.91; found 1811.3.

### Synthesis of Compound 3d

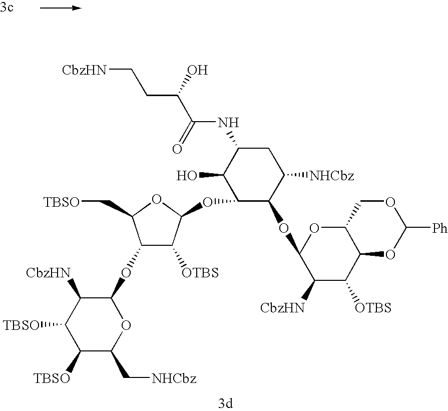

To a stirred solution of benzyloxy 4-hydroxy aminobutric acid (27 mg, 0.11 mmol) and N-hydroxy succinimide (12 mg, 0.11 mmol) in dry THF (2 mL) was added DCC (22 mg, 0.11 mmol) with stirring continued for an additional 1 hour at room temperature. To this reaction mixture the free amine, Compound 3c (95 mg, 0.053 mmol) in dry THF (2 mL) and triethyl amine (15 μL, 0.11 mmol) was added with stirring for 12 hours at room temperature. Evaporation of the solvent followed by purification by flash column chromatography yielded Compound 3d (80 mg, 74%).

[α]_{D}=+19° (c 0.4, CHCl₃).

### Synthesis of Compound 3e

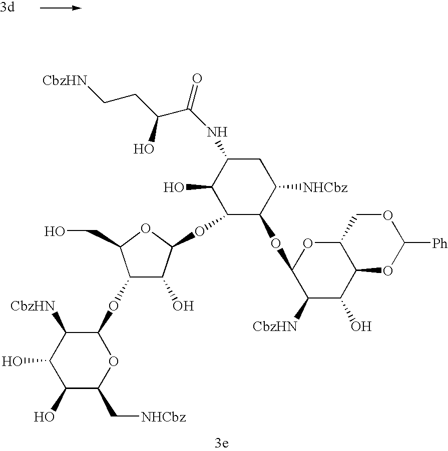

Compound 3d (90 mg, 0.044 mmol) was dissolved in dry pyridine (2 mL), HF.Py (2 mL) was added at 0° C., the reaction was slowly brought to room temperature and stirred for 2 days. Water was added and the reaction mixture was extracted with ethyl acetate followed by washing with brine. The organic layer was dried over Na₂SO₄ and evaporated to give the crude product. The crude product was purified by column chromatography to yield Compound 3e (50 mg, 77%).

[α]_{D}=+20° (c 0.6, CHCl₃). ESI/MS calcd for C₇₄H₈₆N₆O₂₆ (M+H⁺); 1475.56; found 1475.7.

### Synthesis of Compound 3

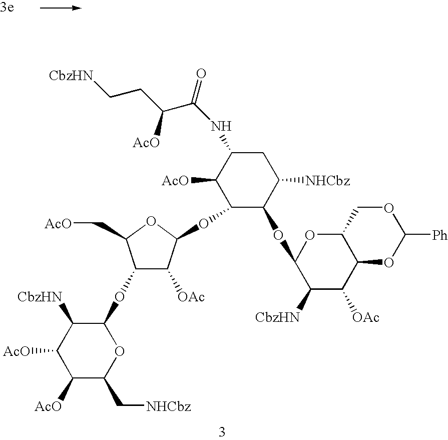

To a solution of Compound 3e (270 mg, 0.183 mmol) in pyridine (2 mL) was added acetic anhydride (1 mL) with stirring maintained for 24 hours at room temperature. Water (10 mL) was added and the precipitated product was filtered. The aqueous layer was extracted with ethyl acetate, washed with saturated CuSO₄, brine and the organic layer was dried over anhydrous Na₂SO₄. The organic layer was combined with the precipitated product and evaporated to provide the crude material, which yielded Compound 3 (300 mg, 93%) after column chromatography.

[α]_{D}=+7.5° (c 0.2, CHCl₃). ESI/MS calcd for C₈₈H₁₀₀N₆O₃₃ (M+H⁺) 1768.63; found 1769.8.

### Example 3

### Synthesis of Compound 4

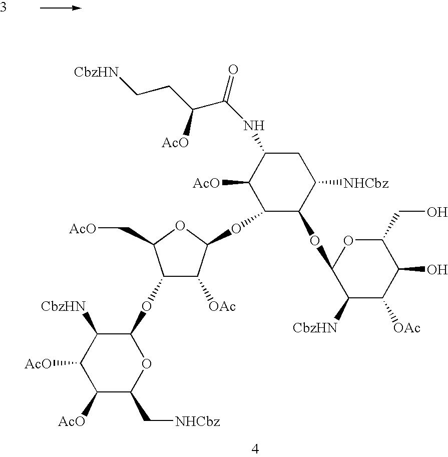

Compound 3 (300 mg, 0.17 mmol) was stirred in 20 mL of acetic acid/water mixture (4:1) at room temperature for 4 days. Water was added and the precipitated product was filtered. The aqueous layer was extracted with ethyl acetate, washed with water, brine and the organic layer was dried over anhydrous Na₂SO₄. The organic layer was combined with the precipitated product and evaporated to yield the crude material, which yielded Compound 4 (280 mg, 98%) after column chromatography.

[α]_{D}=+10.7° (c 0.3, CHCl₃). HRMS calcd for C₈₁H₉₇N₆O₃₃ (M+H⁺) 1681.60911; found 1681.60830.

### Example 4

### Synthesis of Compound 5

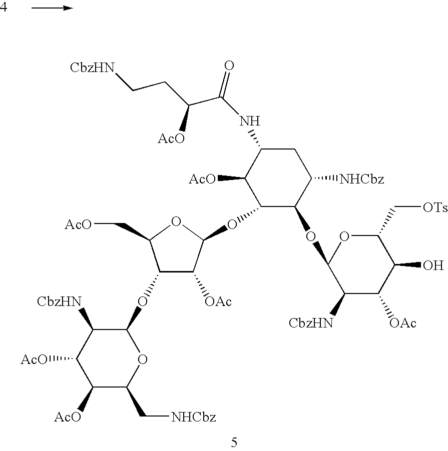

To a solution of Compound 4 (290 mg, 0.17 mmol) in pyridine (2 mL) was added TsCl (36 mg, 0.19 mmol) and DMAP (5 mg, 0.041 mmol) with stirring maintained for 12 hours at room temperature. An additional 1.1 equivalent of TsCl (36 mg, 0.19 mmol) was added and the reaction was stirred for additional 8 hours at room temperature. Water was added and the precipitated product was filtered. The aqueous layer was extracted with ethyl acetate, washed with water, brine and the organic layer was dried over anhydrous Na₂SO₄. The organic layer was combined with the precipitated product and evaporated to yield the crude material. Compound 5 (300 mg, 96%) was obtained after column chromatography.

[α]_{D}=+14.8° (c 0.25, CHCl₃). HRMS calcd for C₈₈H₁₀₂N₆O₃₅S (M+H⁺) 1835.61796; found 1835.61976.

### Example 5

### Synthesis of Compound 6

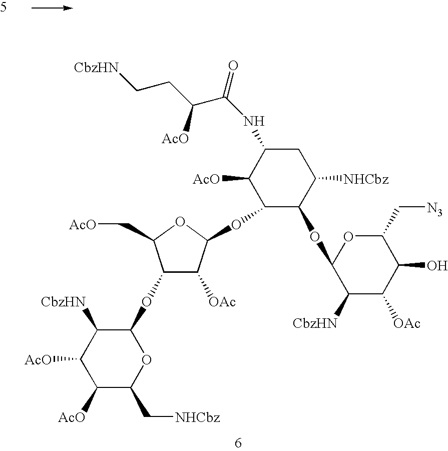

To a solution of Compound 5 (320 mg, 0.175 mmol) in dry DMF (3 mL) was added NaN₃ (113 mg, 1.74 mmol) with stirring maintained for 24 hours at 70° C. Water was added and the resulting mixture was extracted with ethyl acetate followed by washing with water and then brine. The organic layer was dried over anhydrous Na₂SO₄ and evaporated under reduced pressure. Compound 6 (252 mg, 84%) was obtained following column chromatography.

[α]_{D}=+11.3° (c 0.3, CHCl₃). ESI/MS calcd for C₈₁H₉₅N₉O₃₂ (M+H⁺) 1705.61; found 1707.0.

### Example 6

### Synthesis of Compound 7

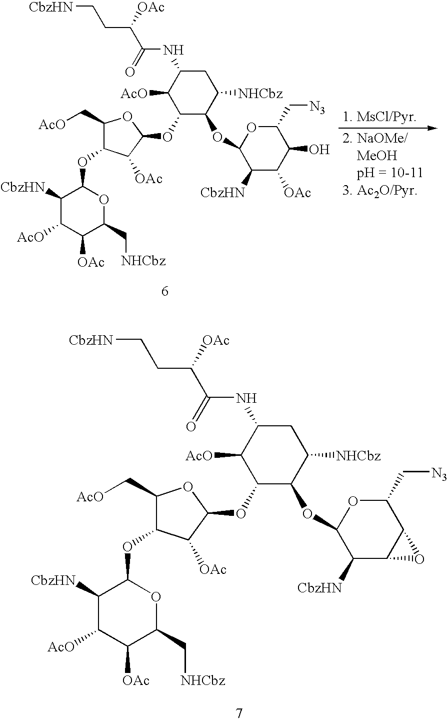

To a stirred solution of Compound 6 (115 mg, 0.067 mmol) in pyridine (2 mL) was added 10 μL of MsCl (0.13 mmol) at 0° C. and the reaction mixture was slowly brought to room temperature and stirred for 3 hours. Few drops of water were added to quench the reaction and extracted with ethyl acetate. The organic layer was washed with saturated CuSO₄, water, brine and dried over anhydrous Na₂SO₄, followed by concentration of the solvent yielding the corresponding crude product. The crude product was dissolved in pre-prepared NaOMe in methanol (pH=10-11) and stirred at room temperature for 12 hours. Dry ice was added to quench the reaction and extracted with ethyl acetate. The organic layer was washed with water, brine and dried over anhydrous Na₂SO₄, followed by concentration of the solvent yielding the corresponding crude product. This material was dissolved in pyridine (2 mL) and acetic anhydride (2 mL) and stirred at room temperature for 12 h. The reaction mixture was extracted with ethyl acetate followed by washing with saturated NaHCO₃, water and brine. Evaporation of the solvent yielded the crude material, which was purified by flash column chromatography to yield Compound 7 (85 mg, 77%).

[α]_{D}=+30.5° (c 0.8, CHCl₃). ESI/MS calcd for C₇₉H₉₁N₉O₃₀ (M+H⁺) 1646.61; found 1647.5.

### Example 7

### Synthesis of Compound 8

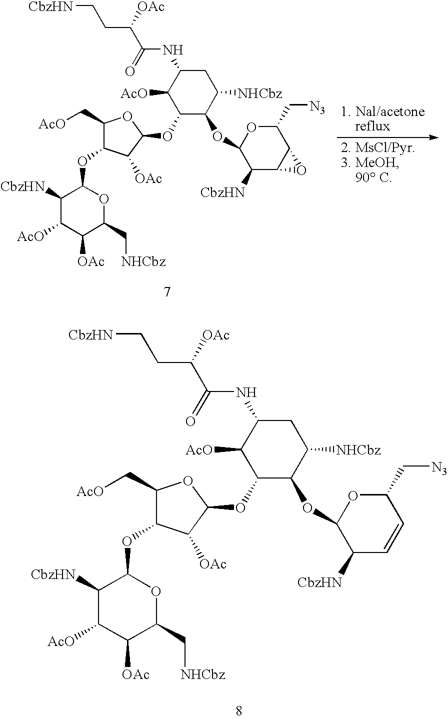

To a stirred solution of Compound 7 (80 mg, 0.049 mmol) in acetone (5 mL) were added NaI (36 mg, 0.24 mmol), NaOAc (2 mg, 0.024 mmol) and 0.1 mL of AcOH and refluxed at 75° C. for 24 hours. Evaporation of the solvent followed by extraction with ethyl acetate and washing with water, saturated NaHCO₃, brine and dried over anhydrous Na₂SO₄. Concentration of the solvent gave the crude product. This was dissolved in pyridine and 7 μL of MsCl was added at 0° C. The reaction mixture was stirred at room temperature for 3 h. Then one drop of methanol was added and heated at 70° C. for 24 h. After usual work up followed by flash column chromatography yielded pure Compound 8 (56 mg, 76%). [α]_{D}=+15.2° (c 0.5, CHCl₃). ESI/MS calcd for C₇₉H₉₁N₉O₂₉ (M+H⁺) 1630.61; found 1631.5.

### Example 8

### Synthesis of Compound 9

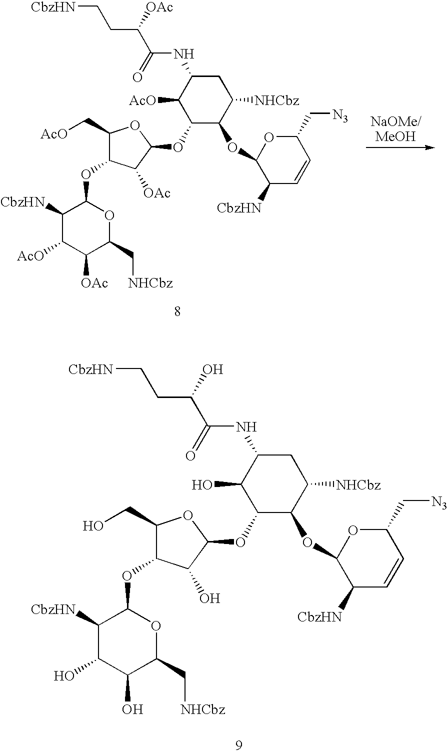

Compound 8 (56 mg, 0.034 mmol) was dissolved in 10 mL of pre-prepared NaOMe in methanol (pH=10-11) and stirred at room temperature for 12 hours. Dry ice was added to quench the reaction and extracted with ethyl acetate. The organic layer was washed with water, brine and dried over anhydrous Na₂SO₄, followed by concentration of the solvent yielding the corresponding crude product. This material was purified by flash column chromatography to yield pure Compound 9 (31 mg, 66%). [α]_{D}=+30.3° (c 1, CHCl₃). ESI/MS calcd for C₆₇H₇₉N₉O₂₃ (M+H⁺) 1378.39; found 1379.1.

### Example 9

### Synthesis of Compound 10 (3′,4′-Di-deoxy-N-1 haba neomycin

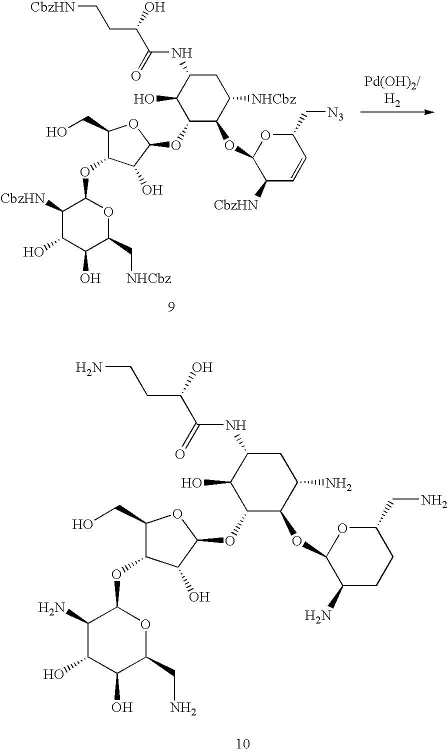

To a stirred solution of Compound 9 (30 mg, 0.022 mmol) in 2 mL of AcOH/water (4:1) mixture was added 20% Pd(OH)₂ (30 mg) and stirred under hydrogen atmosphere using hydrogen balloon for 3 hours. Filteration over celite followed by lypholyzation yielded Compound 10 (20 mg, 93%). [α]_{D}=+10.1° (c 0.3, H₂O). ¹H NMR (400 MHz, D₂O) δ 5.7 (br s, 1H), 5.22 (s, 1H), 5.11 (br s, 1H), 4.33 (t, J=5.8 Hz, 1H), 4.25-4.2 (m, 1H), 4.14-4.10 (m, 2H), 4.05-3.95 (m, 2H), 3.9-2.8 (m, 17H), 2.1-1.9 (m, 4H), 1.82-1.6 (m, 4H).; ¹³C NMR (125 MHz, D₂O) δ 176.4, 111.1, 96.1, 95.2, 86.5, 82.0, 76.0, 74.4, 74.3, 74.1, 70.9, 70.3, 68.4, 68.1, 66.7, 61.1, 51.6, 49.8, 49.4, 43.3, 41.1, 37.4, 37.2, 31.6, 30.6, 26.2, 21.

ESI/MS calcd for C₂₇H₅₃N₇O₁₃ (M+H⁺) 683.75 (M+H⁺); found: 684.6.

### Example 10

### Synthesis of Compound 11

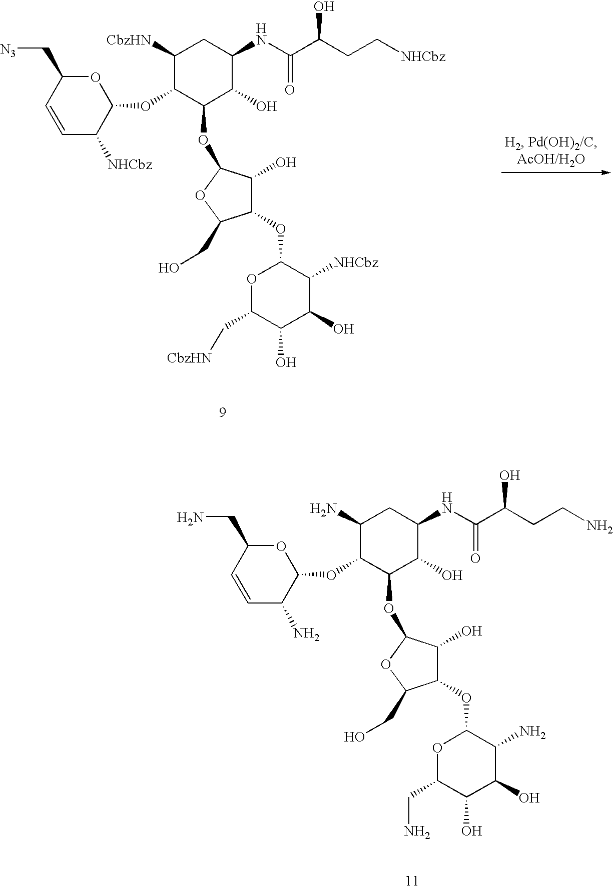

Compound 9 (50 mg) was dissolved in 2 mL 80% AcOH/water (v/v) and 10 mg palladium hydroxide on carbon (20% Pd) was added. The reaction was stirred under 1 atm hydrogen at room temperature, monitoring closely by LC/MS. The stirring was periodically stopped while awaiting LC/MS data. The reaction was adjudged to be complete when the majority of the benzyl carbamates had been deprotected, but the double bond had not been fully reduced. At this point there was an approximately 1:1 ratio (by mass spec) of reduced to unreduced double bond.

The catalyst was removed by filtration and washed with water, and the combined washings dried on the lyophilizer. The resulting solid was taken up in water, basified with aqueous ammonia and purified by reverse-phase HPLC. 2 mg of Compound 11 was obtained. ESI/MS calcd for C₂₇H₅₁N₇O₁₃ 682.4 (M+H⁺); found: 682.2.

### Example 11

### Synthesis of Compound 13 (4′,6′-Dichloro-hexa-O-benzoyl penta-N-benzyloxycarbonyl paromomycin)

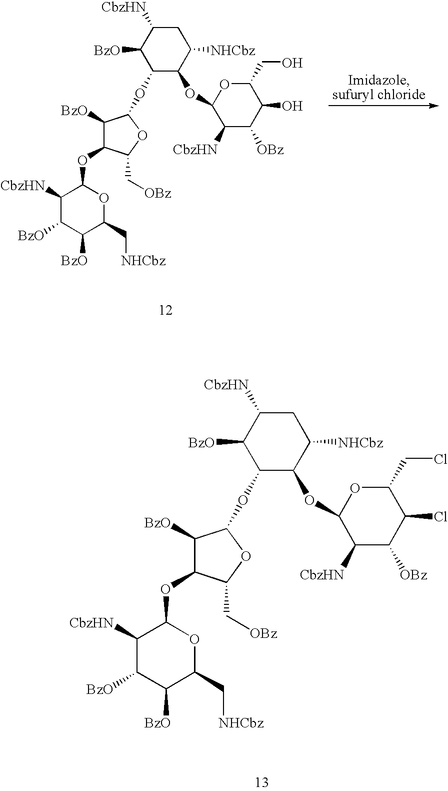

Compound 12 was prepared according to the process disclosed in Hanessian, S.; Vatele, J. M., J. Antibiotics, 1980, 33(6), 675-8. To a stirred solution of Compound 12 (2.55 g, 1.34 mmol) and 5.17 g (75 mmol, 56 eq.) imidazole in 26 ml dry DMF was added 2.57 ml (30.82 mmol, 23 eq.) sulfuryl chloride at −40° C. dropwise. The reaction mixture was stirred for 1 hour and an additional 2 days at room temperature before it was poured into saturated NaHCO₃ solution. The layers were separated and the organic layer was concentrated in vacuo. The crude material was purified by flash column chromatography to yield the pure Compound 13 (2.4 g, 1.23 mmol, 92%). [α]_{D}²⁵: 76.06 (c=3.4, CHCl₃). MS (ESI): m/z=1947.0 [M+H]⁺ calcd. for C₁₀₅H₉₇Cl₂N₅O₂₈ 1947.58.

### Example 12

### Synthesis of Compound 14 (6′-Azido-4′-deoxy-hexa-O-benzoyl penta-N-benzyloxycarbonyl paromomycin

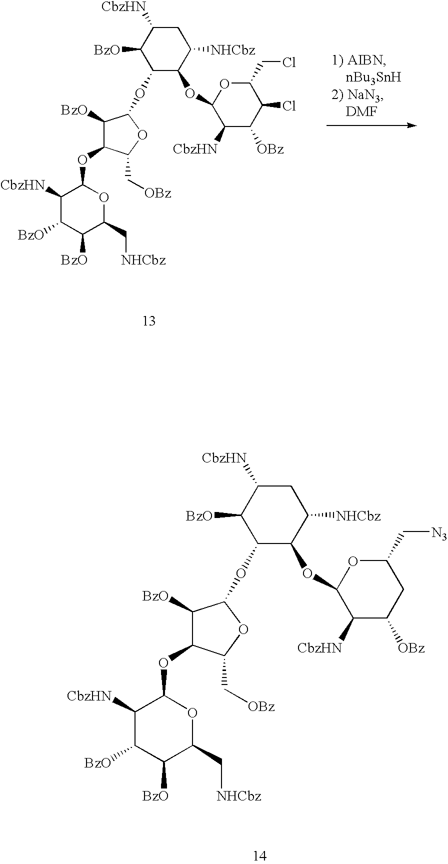

To a stirred solution of Compound 13 (1.8 g, 0.923 mmol) and 35 mg AIBN in 37 ml dry toluene was added 0.94 ml (3.5 mmol, 3.79 eq.) tributyltin hydride. The reaction mixture was refluxed for 2 hours. After evaporation of the solvent and flash column chromatography pure 6′-Chloro-4′-deoxy-hexa-O-benzoyl penta-N-benzyloxycarbonyl paromomycin (1.7 g, 0.89 mmol, 96%) was obtained. [α]₂^{D}5: 57.65 (c=2.0, CHCl₃). LCMS calcd. for C₁₀₅H₉₉ClN₅O₂₈ (M+H⁺): 1912.62, 1914.62: found 1912.3, 1914.4.

The obtained 6′-Chloro-4′-deoxy-hexa-O-benzoyl penta-N-benzyloxycarbonyl paromomycin (1.6 g, 0.837 mmol) and sodium azide (113 mg, 1.67 mmol, 2 eq.) were dissolved in 40 ml dry DMF and stirred at 90° C. for 2 days. The reaction mixture was concentrated in vacuo and flash column chromatography yielded pure Compound 14 (1.2 g, 0.626 mmol, 75%). [α]_{D}²⁵: 68.3 (c=2.0, CHCl₃). IR (CHCl₃, NaCl): 2200 cm⁻¹.

### Example 13

### Synthesis of Compound 15 (6′-Azido-4′-deoxy-3′,2″,5″,3′″,4′″-penta-O-tert-butyl dimethyl silanyloxy penta-N-benzyloxycarbonyl paromomycin)

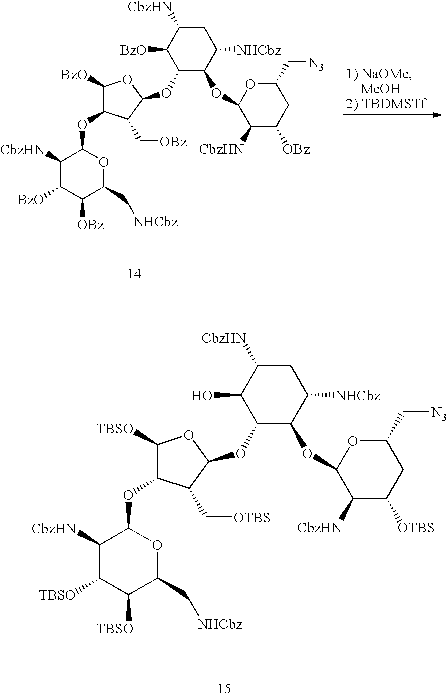

Compound 14 (1.2 g, 0.625 mmol) was dissolved in 40 ml of a methanolic sodium methoxide solution (pH=9) and stirred at room temperature. After 5 days the reaction mixture concentrated in vacuo and following flash column chromatography yielded the pure 6′-Azido-4′-deoxy-penta-N-benzyloxycarbonyl paromomycin (0.548 g, 0.422 mmol, 67%). [α]_{D}²⁵46.7 (c=2.0, CHCl₃). MS (ESI): m/z=1667.3 [M+H]⁺ calcd. for C₉₃H₁₄₅N₈O₂₂Si₅ 1867.5.

To a stirred solution of the obtained 6′-Azido-4′-deoxy-penta-N-benzyloxycarbonyl paromomycin (400 mg, 0.308 mmol) in dry dichloromethane (10 ml) were added 2,4,6-collidine (0.407 ml, 3.08 mmol, 10 eq.) and TBSOTf (0.64 ml, 2.77 mmol, 9 eq.) at 0° C. Then the reaction mixture was slowly brought to room temperature and stirred for 12 hours. Few drops of water were added to quench the excess TBSOTf, followed by extraction with dichloromethane. The organic layer was washed with brine and dried over Na₂SO₄, followed by concentration of the solvent. The crude product was purified by flash column chromatography to yield pure Compound 15 (0.315 g, 0.169 mmol, 55%). [α]_{D}²⁵: 21.15 (c=2.7, CHCl₃). MS (ESI): m/z=1868.1 [M+4H]⁺ calcd. for C₉₃H₁₄₄N₈O₂₂Si₅ (M+4H⁺) 1868.93.

### Example 14

### Synthesis of Compound 16

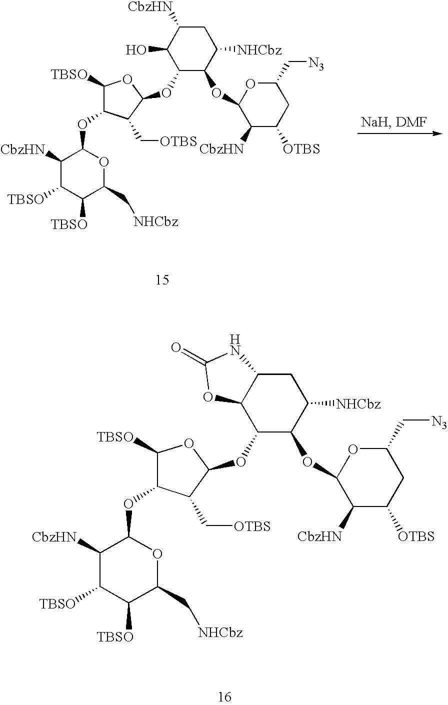

To a stirred solution of Compound 15 (315 mg, 0.169 mmol) in dry DMF (4 ml) was added 60% NaH in mineral oil (7.7 mg, 0.169 mmol) at 0° C. and continued to stir for additional 6 hours. Few drops of saturated ammonium chloride solution were added, followed by extraction with ethyl acetate. The organic layer was washed with saturated brine and dried over anhydrous Na₂SO₄, followed by concentration of the solvent in vacuo. The crude material was purified by flash column chromatography to yield Compound 16 (141 mg, 0.0802 mmol, 47%). [α]_{D}²⁵: 33.64 (c=1.1, CHCl₃). MS (ESI): m/z=1760.1 [M+H]⁺ calcd. for C₈₆H₁₃₈N₈O₂₁Si₅: 1759.89.

### Example 15

### Synthesis of Compound 17 (6′-Azido-4′-deoxy-3′,2″,5″,3′″,4′″-penta-O-tert-butyl dimethyl silanyloxy tetra-N-benzyloxycarbonyl N-1 haba paromomycin)

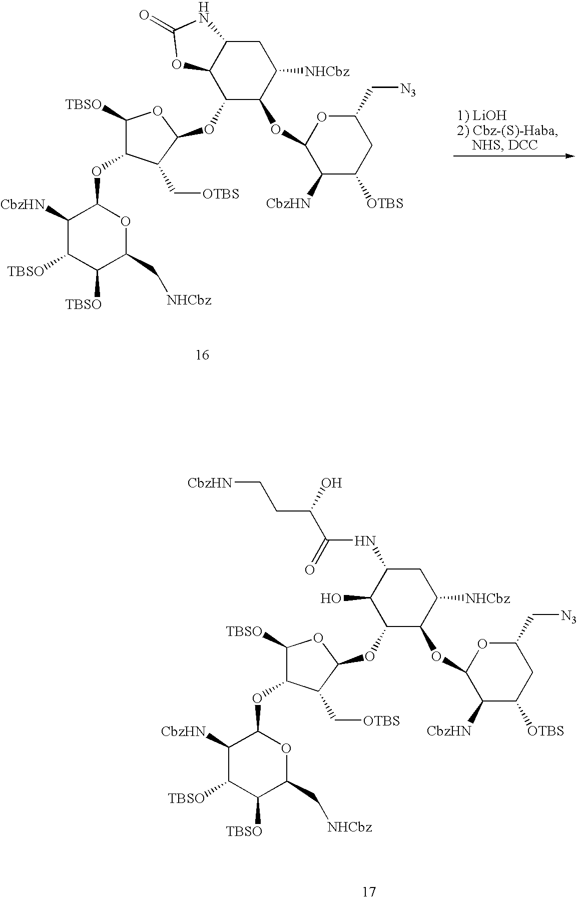

To a stirred solution of Compound 16 (141 mg, 0.0802 mmol) in 3 ml DMF was added 0.5 ml of an aqueous LiOH solution (6 mg, 0.160 mmol, 2 eq.) and continued to stir for additional 3 hours at room temperature. Few drops of saturated ammonium chloride solution were added, followed by extraction with ethyl acetate. The organic layer was washed with brine and dried over Na₂SO₄, followed by concentration of the solvent ion vacuo. The crude product was used for the next step without further purification.

To a stirred solution of benzyloxy 4-hydroxyamino butyric acid (98 mg, 0.4 mmol, 5 eq.) and N-hydroxy succinimide (43 mg, 0.4 mmol, 5 eq.) in dry THF (7 ml) was added DCC (80 mg, 0.4 mmol, 5 eq.) and continued to stir for additional 1 hour at room temperature. To this reaction mixture the above synthesized crude product (0.0802 mmol) in dry THF (7 ml) and triethyl amine (54 μl, 0.4 mmol, 5 eq.) were added and stirred for 12 hours at room temperature. Evaporation of the solvent followed by flash chromatography yielded Compound 17 (116 mg, 0.059 mmol, 74%). [c]_{D}²⁵: 10.14 (c=0.7, CHCl₃). MS (ESI): m/z=1969.1 [M+3H]⁺ calcd. for C₉₇H₁₅₄N₉O₂₄Si₅ 1968.99.

### Example 16

### Synthesis of Compound 18 (4′-Deoxy-N-1 haba neomycin)

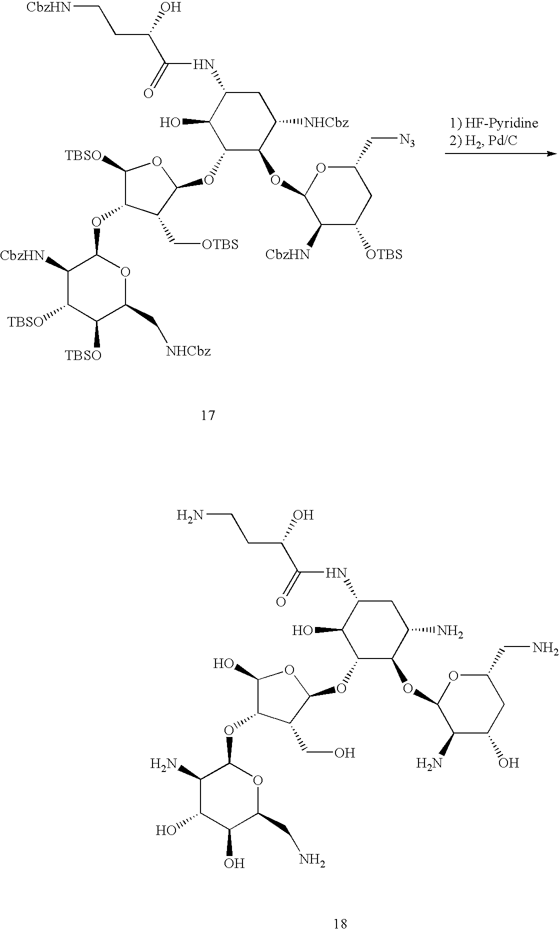

Compound 17 (55 mg, 0.028 mmol) was dissolved in dry pyridine (1.3 ml) and cooled to 0° C. Then HF-Pyridine (1.3 ml) was added dropwise and the reaction was slowly brought to room temperature and stirred for 2 days. Sodium bicarbonate was added carefully to the reaction mixture followed by extraction with ethyl acetate. The organic layer was dried over Na₂SO₄ and concentrated in vacuo. Flash chromatography yielded pure 6′-Azido-4′-deoxy-tetra-N-benzyloxycarbonyl N-1 haba paromomycin (12 mg, 0.0086 mmol, 31%). [α]_{D}²⁵: 16.7 (c=0.6, MeOH). LCMS calcd for C₆₇H₈₂N₉O₂₄ (M+H⁺): 1396.54; found 1397.2.

The obtained 6′-Azido-4′-deoxy-tetra-N-benzyloxycarbonyl N-1 haba paromomycin (8 mg, 0.0057 mmol) was dissolved in 1 ml of a 80% acetic acid solution. 4 mg of palladium hydroxide on charcoal (10%) were added and the reaction mixture was stirred under hydrogen atmosphere (balloon) for 2 hours, then filtered over celite and lypholized to give pure Compound 18 (4 mg, 0.0057 mmol, quant.). [α]_{D}²⁵: 38.75 (c 0.4=, H₂O). MS (ESI): m/z=700.6 [M+H]⁺ calcd for C₂₇H₅₄N₇O₁₄ 700.37. ESI-HRMS: 700.37233; found: 700.37219.

¹H NMR (400 MHz, D₂O) δ 5.90 (s, 1H), 5.28 (s, 1H), 5.16 (s, 1H), 4.40 (m, 1H), 4.28 (m, 1H), 4.09 (m, 5H); 3.86-3.62 (m, 6H), 3.51-3.44 (m, 2H), 3.33-3.14 (m, 5H), 3.01 (m, 3H), 2.08-1.98 (m, 3H), 1.82 (s, 18H), 1.66-1.33 (m, 3H), 1.10 (m, 1H).

### Example 17

### Preparation of tert-butyl-3-oxocyclobutylcarbamate

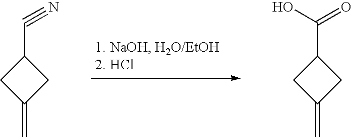

A solution of 70 g of KOH in 500 ml of mixture EtOH/H₂O (1/1, v/v) was added to a 2 L round bottom flask equipped with magnetic stir bar and condenser followed by 3-methylenecyclobutane carbonitrile (Maybrige) (25 g, 0.26 mol). The reaction mixture was refluxed with stirring in an oil bath for 5-6 hours. The reaction was monitored for completion by TLC. Upon completion of reaction, the mixture was cooled and acidified with HCl to a pH of 3-4. Ethanol was evaporated, and the remaining aqueous layer was extracted with 200 mL of Et₂O. Combined organics were washed with water (2×20 mL) followed by brine (once by 30 ml). Organics were dried over Na₂SO₄, filtered and evaporated. The resulting product, 3-methylenecyclobutane carboxylic acid (shown above), was used without further purification in the next step.

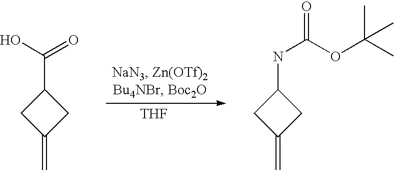

1.0 g (8.9 mmol) of 3-methylenecyclobutane carboxylic acid, 2.0 g (31.1 mmol) of NaN₃, 0.48 g (1.5 mmol) of tetrabutyl ammonium bromide, 0.1 g (0.3 mmol) of Zn(OTf)₂ and 90 ml of dry THF were added to a 250 mL round bottom flask and warmed to 40° C. When the reaction mixture reached this temperature, 2.1 g (9.8 mmol) of Boc₂O was added at once and allowed to react overnight at 45° C. The reaction mixture was then cooled in ice bath. 180 mL of 10% NaNO₂ solution was added and THF was evaporated off. 180 mL EtOAc was used for extraction and the organic layer was washed with 5% NaHCO₃ (2×20 mL) followed by brine (once by 30 ml). Dried organic layer over Na₂SO₄ and evaporated solvent to get a yellow solid. The product was purified on 40 gram Si column to use hexanes/ethyl acetate as eluent, gradient: 0-90% for 1 hour to give 0.57 g of 1-(N-Boc amino)-3-methylenecyclobutane.

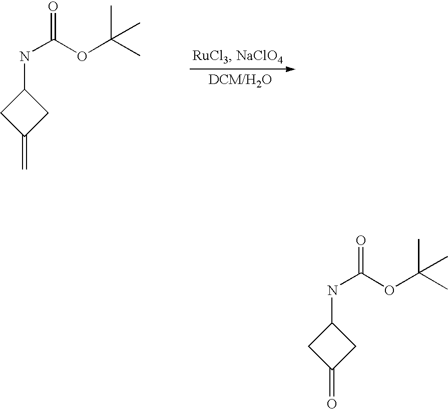

To a 1 L round bottom flask with magnetic stir bar 9.8 g, 53.5 mmol of 1-(N-Boc amino)-3-methylenecyclobutane and 160 mL each of DCM and water were added. This mixture was stirred vigorously until alkene had dissolved. Next, 3 g, 21.7 mmol of K₂CO₃ was added to stirring mixture, followed by 35 g, 163.5 mmol of NaClO₄, 0.2 g, 0.72 mmol of tetrabutylammonium chloride, and 0.6 g, 7.6 mmol of RuCl₃. The reaction vessel was closed and allowed to stir vigorously at ambient temperature. The reaction was monitored by TLC, 70/30 (v/v) hexanes/ethyl acetate. Upon completion of reaction, reaction mixture was filtered through a pad of celite to remove solids. Filtrate was transferred to a separation funnel, and aqueous layer was extracted twice with 50 mL of DCM was washed with 5% NaHCO₃ (2×30 mL) followed by brine (once by 30 ml) and dried over Na₂SO₄. The organics were then filtered and evaporated to yield tert-butyl-3-oxocyclobutylcarbamate. The final product was purified by flash chromatography on Si gel using a 120 gram column and large cartridge. The solvent system used was ethyl acetate/hexanes, 0%-60% ethyl acetate over one hour gradient.

### Example 18

### General Procedure for Synthesis of Alpha-hydroxy carboxylic Acids

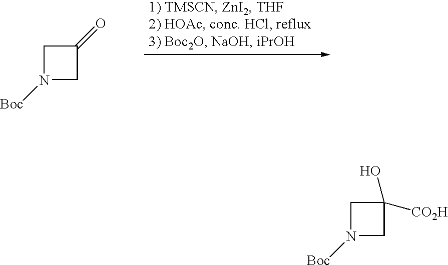

Step 1. O-(Trimethylsilyl) cyanohydrines

A 50-mL, one-necked flask equipped with a magnetic stirring bar and drying tube was charged with 10 mmol of ketone or aldehyde (such as N-Boc-3-Pyrrolidonone, N-Boc-3-Azetidinone, N-Boc-4-piperidone, N-Boc-3-azetidincarbox aldehyde, or tert-butyl-3-oxocyclobutylcarbamate), 1.39 g, 14 mmol of trimethylsilyl cyanide (Aldrich), 90 mg (0.28 mmol) of anhydrous zinc iodide, and 50 mL of dry THF. The solution was stirred at room temperature for 24 hours. The solvent was removed on a rotary evaporator; the residue was taken in 60 ml of EtAc. The organic layer was washed, sequentially, with 5% NaHCO₃ (2×30 mL), H₂O (1×30 mL), brine (1×30 mL) and dried over anhydrous Na₂SO₄. The solvent was evaporated off and the residue was used in the next step without purification.

Step 2. Acid hydrolysis to α-hydroxy carboxylic Acid

AcOH (25 ml) and conc. HCl (25 ml) were added to the unpurified material from Step 1 and the reaction mixture was refluxed 2-3 hours. The reaction mixture was concentrated to dryness to give a white solid. The solid was used in the next step without purification.

Step 3. Boc Protection

20 ml of 2 M NaOH solution and 20 ml of i-PrOH were added to the solid from Step 2. The flask was put in an ice-bath and Boc₂O (6.6 g, 3 mmol) was added in several portions. The reaction mixture was then stirred for 4 hours at room temperature. After stirring at room temperature, i-PrOH was evaporated off, 50 ml of H₂O was added, and the basic aqueous phase was extracted by Et₂O (2×30 ml). After extraction with ether, the aqueous phase was made acidic (pH=3) by diluted H₃PO₄ and was extracted by EtOAc (2×60 ml). Organic phase was washed, sequentially H₂O (2×30 mL), brine (1×30 mL) and dried over anhydrous Na₂SO₄ Organic phase was concentrated to give pure N-Boc-α-hydroxy carboxylic acids. Yields varied from 56-72%.

### Example 19

### General Procedure for Synthesis of Representative Aminoglycoside Compounds

Representative aminoglycoside compounds of formula I may be prepared using various alpha-hydroxy carboxylic acids (such as, for example, the N-Boc-alpha-hydroxy carboxylic acid as prepared according to the general procedure of Example 18) as follows:

Procedure 1

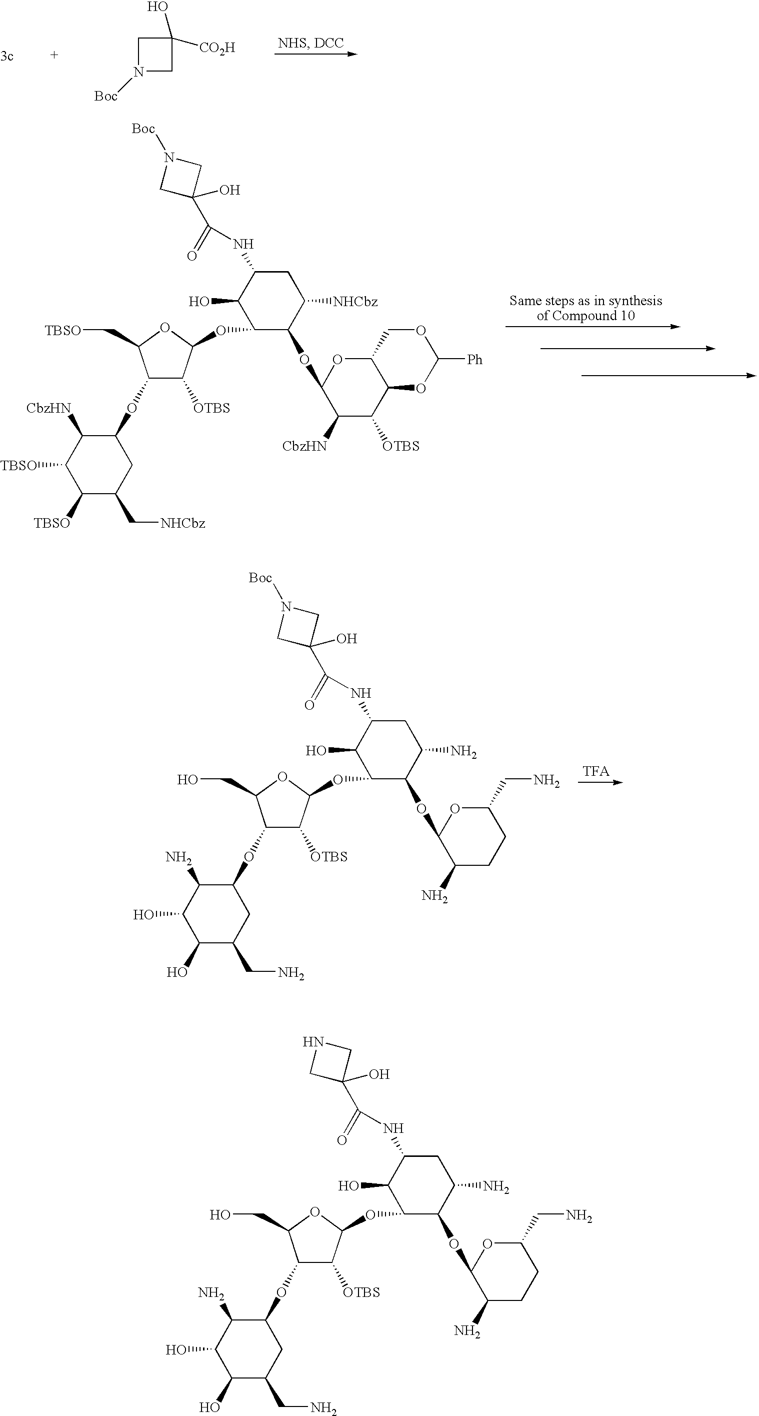

Procedure 2

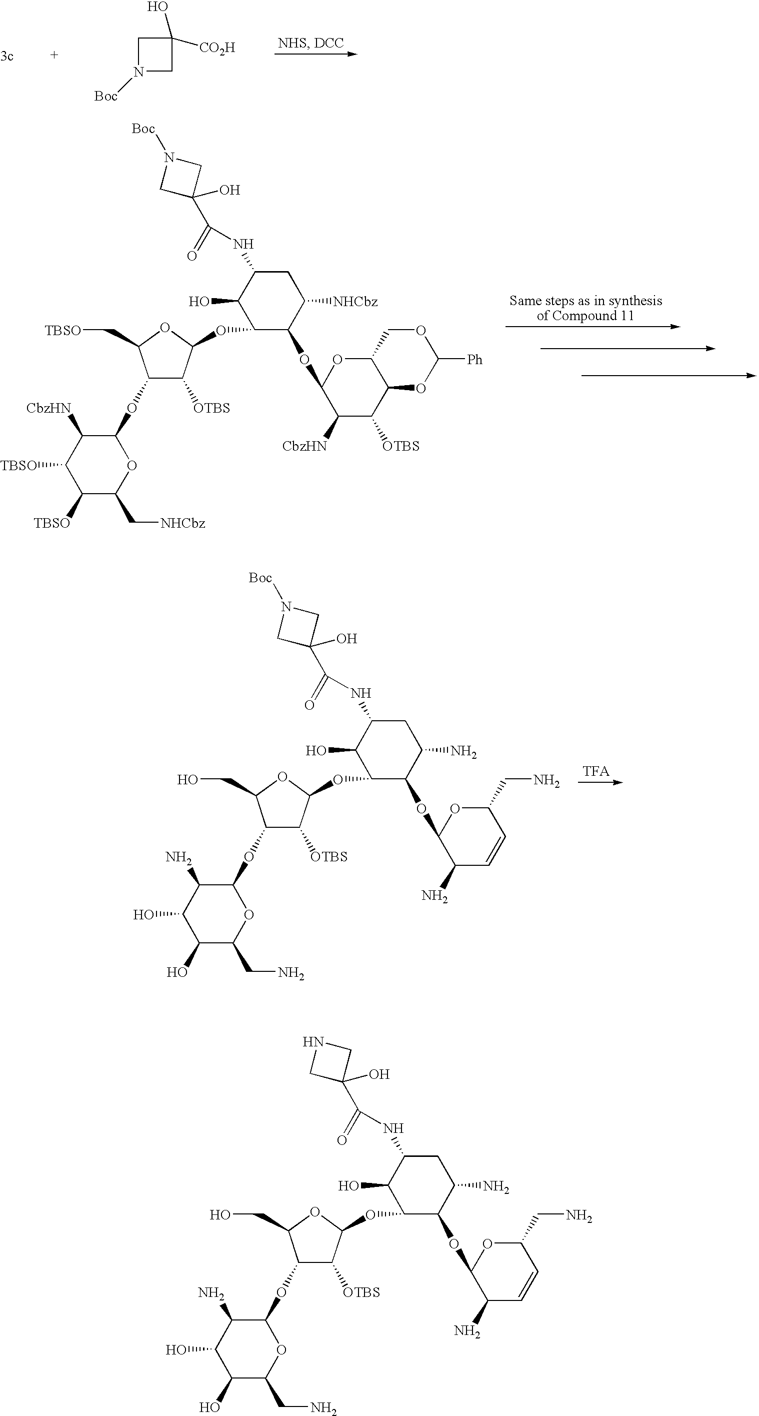

Procedure 3

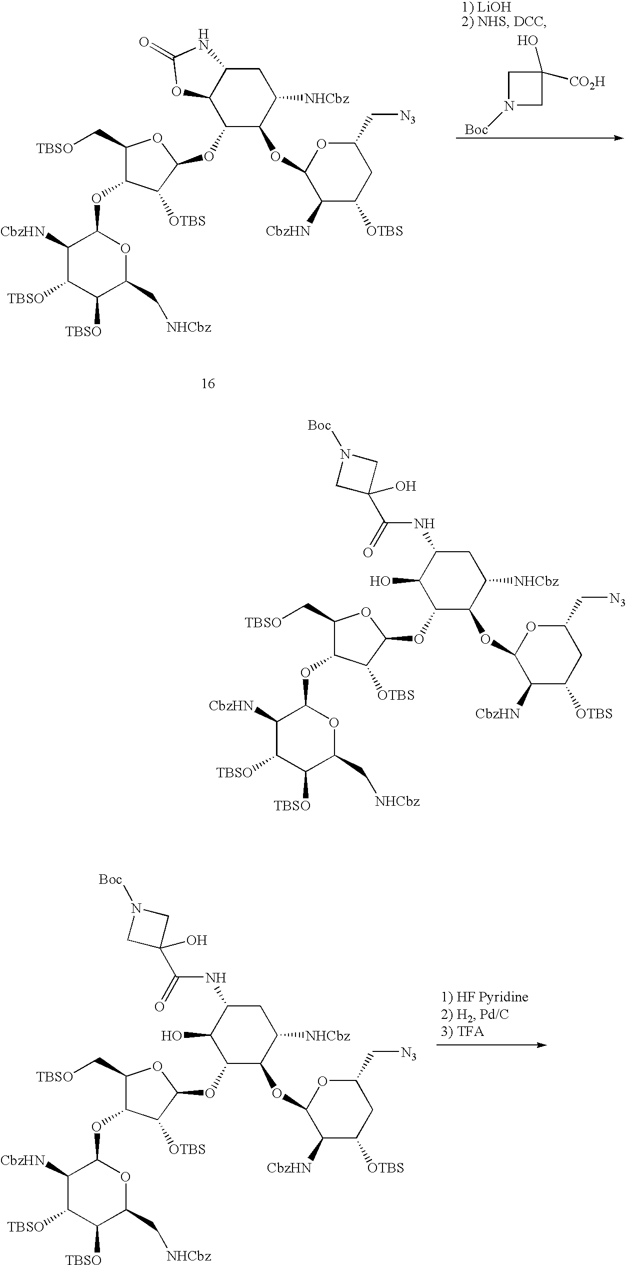

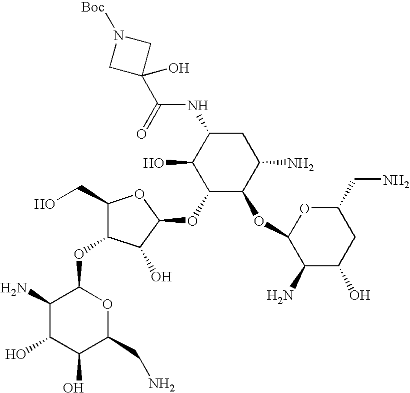

### Example 20

### In Vitro Antibacterial Activity Determination of Minimum Inhibitory Concentrations (MICs)

The MIC assays were carried out in 150 μL volume in duplicate in 96-well clear flat-bottom plates. The bacterial suspension from an overnight culture growth in appropriate medium was added to a solution of test compound in 4% DMSO in water. Final bacterial inoculum was approximately 10⁵-10⁶ CFU/well. The percent growth of the bacteria in test wells relative to that observed for a well containing no compound was determined by measuring absorbance at 595 nm (A₅₉₅) after 24 h. The MIC was determined as a range of single compound where the complete inhibition of growth was observed at the higher concentration and cells were viable at the lower concentrations. Both ampicillin and tetracycline are used as antibiotic-positive controls in each screening assay for E. coli, S. aureus and K. pneumoniae. Ciprofloxacin is used as an antibiotic-positive control in each screening assay for P. aeruginosa. Amikacin is used as an antibiotic-positive control in each screening assay for A. baumannii. Data for certain representative compounds is shown in Table 1 below. Each of the bacterial cultures that are available from ATCC (www.atcc.org) is identified by its ATCC number.

TABLE 1

| | MIC (μg/ml) | | | | |
|---|---|---|---|---|---|
| | | | P. | K. | A. |
| | E. coli | S. aureus | aeruginosa | pneumoniae | baumannii |
| Compound # | ATCC 25922 | ATCC 29213 | ATCC 27853 | ATCC 10031 | ATCC 19606 |
| Ciprofloxacin | A | | A | | |
| Amikacin | B | B | B | A | C |
| 10 | A | A | A | A | B |
| 11 | B | A | B | A | B |
| 18 | B | B | A | A | B |

* MIC Key:
MIC's of 1.0 μg/mL or less = A
MIC's of greater than 1.0 μg/mL to 8.0 μg/mL = B
MIC's of greater than 8.0 μg/mL = C

### Example 21

### In Vitro Antibacterial Activity Determination of Minimum Inhibitory Concentrations (MICs) Against Aminoglycoside-Resistant Pseudomonas Aeruginosa

MIC assays were carried out as set forth in Example 20 above against certain aminoglycoside-resistant strains of Pseudomonas Aeruginosa. Data for certain representative compounds is set forth in Table 2 below. As noted, Compound 11 showed superior activity on certain strains of aminoglycoside-resistant Pseudomonas aeruginosa, particularly those strains expressing efflux-based resistance alone or in combination with aminoglycoside modifying enzymes (AMEs), in comparison to compound 10 and comparative compounds A and B.

TABLE 2

| Compound | MIC (μg/ml) | | | | |
|---|---|---|---|---|---|
| # | APAE040 | APAE042 | APAE1009 | APAE1058 | APAE1068 |
| 10 | 2 | 1 | 4 | 32 | 8 |
| 11 | 1 | 0.5 | 2 | 16 | 2 |
| Comparative | 4 | 4 | >8 | >8 | 8 |
| Cpd A | | | | | |
| Comparative | 1 | 2 | >8 | >8 | 4 |
| Cpd B | | | | | |

| Strain | ACH Code | Origin | Phenotype |
|---|---|---|---|
| P. aeruginosa | APAE040 | SPRI | AAC(3)-I |
| P. aeruginosa | APAE042 | SPRI | AAC(6′)-II |
| P. aeruginosa | APAE1009 | RJ - Greece | AAC(6′)-I + Efflux |
| P. aeruginosa | APAE1058 | CF - USA | High level efflux |
| P. aeruginosa | APAE1068 | Focus - France | AAC(6′)-I + Efflux |

*Key:
Comparative compound A is 3′,4′-Di-deoxy-3′,4′-Di-dehydro-neomycin-B
Comparative compound B is 3′,4′-Di-deoxy-neomycin-B

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A compound having the following formula II: 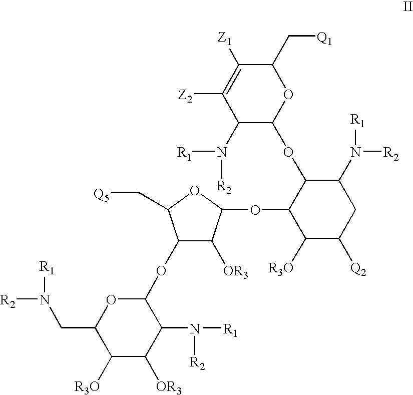
or a stereoisomer, prodrug or pharmaceutically acceptable salt thereof,
wherein:
Q₁ is —OH, a protected hydroxyl, amino or a protected amino group;
Q₂ is 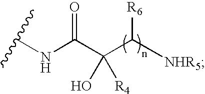
Q₅ is —OH, a protected hydroxyl, amino or a protected amino group;
each R₁ and R₂ is, independently, H or an amino protecting group;
each R₃ is, independently, H or a hydroxyl protecting group;
each R₄, R₅ and R₆ is, independently, H or C₁-C₆ alkyl, or R₄ and R₅ together with the atoms to which they are attached can form a carbocyclic or heterocyclic ring having from 4 to 6 ring atoms, or R₅ and R₆ together with the atoms to which they are attached can form a carbocyclic or heterocyclic ring having from 4 to 6 ring atoms, or R₄ and R₆ together with the atoms to which they are attached can form a carbocyclic ring having from 4 to 6 ring atoms;
n is an integer from 1 to 3; and
each Z₁ and Z₂ is, independently, H, —OH or a protected hydroxyl, and
wherein (i) at least one of Z₁ and Z₂ is H, and (ii) when Q₁ is —OH or a protected hydroxyl then Z₁ is H.

2. The compound of claim 1 wherein each R₁, R₂ and R₃ are H.

3. The compound of claim 2 wherein Q₅ is amino.

4. The compound of claim 3 wherein Q₁ is amino.

5. The compound of claim 4 wherein Q₂ is:

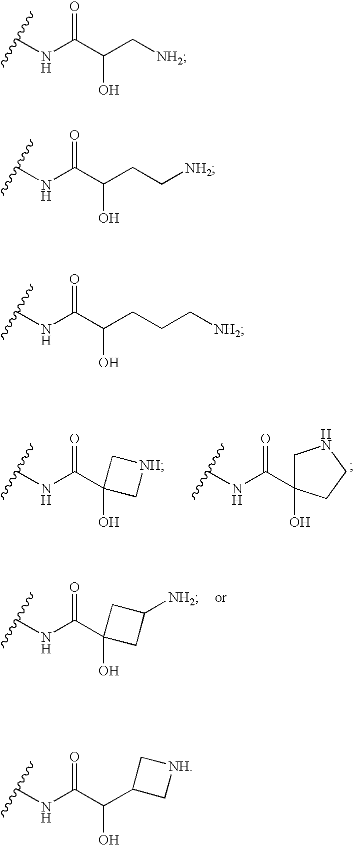

6. The compound of claim 5 wherein Z₁ and Z₂ are H.

7. The compound of claim 5 wherein Z₁ is H and Z₂ is —OH.

8. The compound of claim 5 wherein Z₁ is —OH and Z₂ is H.

9. The compound of claim 3 wherein Q₁ is —OH.

10. The compound of claim 9 wherein Q₂ is:

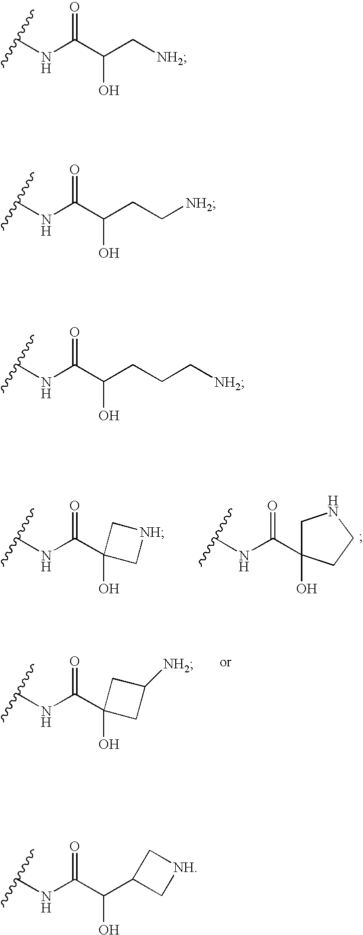

11. The compound of claim 10 wherein Z₁ and Z₂ are H.

12. The compound of claim 10 wherein Z₁ is H and Z₂ is —OH.

13. The compound of claim 2 wherein Q₅ is —OH.

14. The compound of claim 13 wherein Q₁ is amino.

15. The compound of claim 14 wherein Q₂ is:

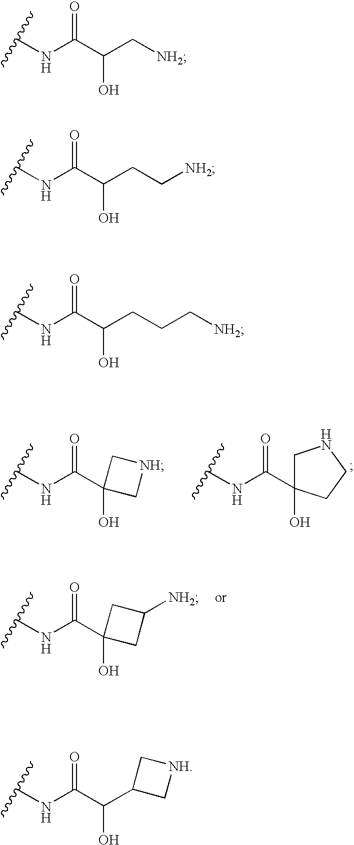

16. The compound of claim 15 wherein Z₁ and Z₂ are H.

17. The compound of claim 15 wherein Z₁ is H and Z₂ is —OH.

18. The compound of claim 15 wherein Z₁ is —OH and Z₂ is H.

19. The compound of claim 13 wherein Q₁ is —OH.

20. The compound of claim 19 wherein Q₂ is:

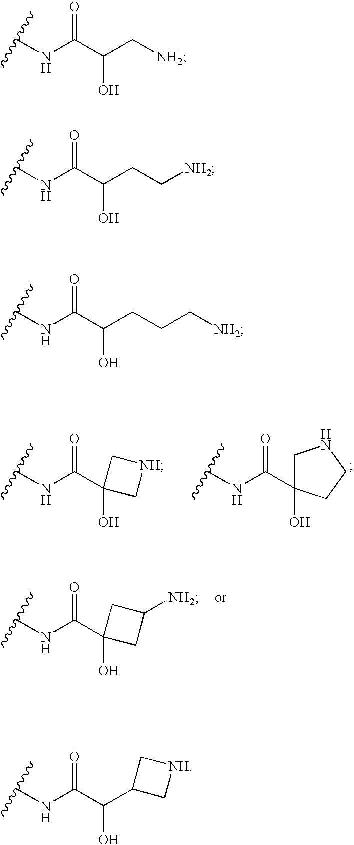

21. The compound of claim 20 wherein Z₁ and Z₂ are H.

22. The compound of claim 20 wherein Z₁ is H and Z₂ is —OH.

23. The compound of claim 1 having the configuration:

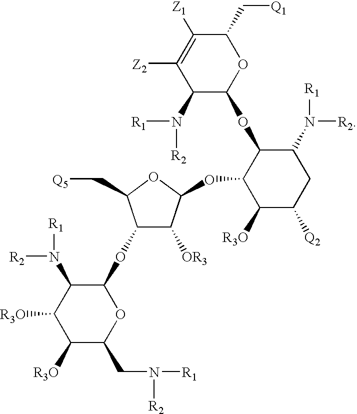

24. A pharmaceutical composition comprising a compound of claim 1, or a stereoisomer, pharmaceutically acceptable salt or prodrug thereof, and a pharmaceutically acceptable carrier, diluent or excipient.

25. A method of treating a bacterial infection in a mammal comprising administering to the mammal an effective amount of a compound of claim 1.

26. A compound having the following formula III: 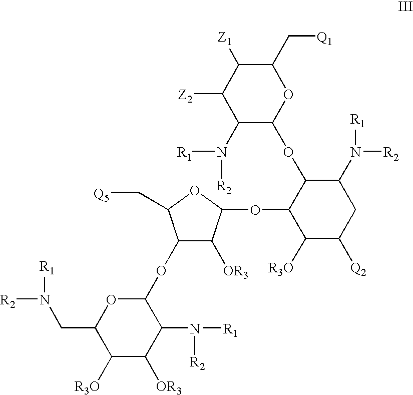
or a stereoisomer, prodrug or pharmaceutically acceptable salt thereof,
wherein:
Q₁ is —OH, a protected hydroxyl, amino or a protected amino group;
Q₂ is 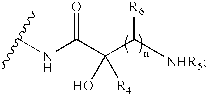
Q₅ is —OH, a protected hydroxyl, amino or a protected amino group;
each R₁ and R₂ is, independently, H or an amino protecting group;
each R₃ is, independently, H or a hydroxyl protecting group;
each R₄, R₅ and R₆ is, independently, H or C₁-C₆ alkyl, or R₄ and R₅ together with the atoms to which they are attached can form a carbocyclic or heterocyclic ring having from 4 to 6 ring atoms, or R₅ and R₆ together with the atoms to which they are attached can form a carbocyclic or heterocyclic ring having from 4 to 6 ring atoms, or R₄ and R₆ together with the atoms to which they are attached can form a carbocyclic ring having from 4 to 6 ring atoms;
n is an integer from 1 to 3; and
each Z₁ and Z₂ is, independently, H, —OH or a protected hydroxyl, and
wherein (i) one of Z₁ and Z₂ is H, and (ii) when Q₁ is —OH or a protected hydroxyl then Z₁ is H.

27. The compound of claim 26 wherein each R₁, R₂ and R₃ are H.

28. The compound of claim 27 wherein Q₅ is amino.

29. The compound of claim 28 wherein Q₁ is amino.

30. The compound of claim 29 wherein Q₂ is:

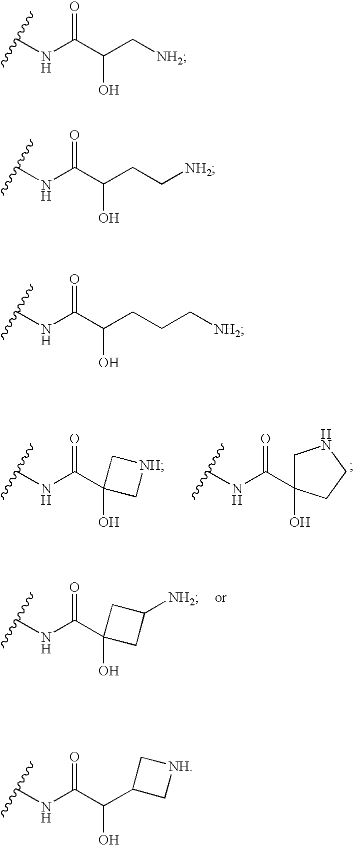

31. The compound of claim 30 wherein Z₁ is H and Z₂ is —OH.

32. The compound of claim 30 wherein Z₁ is —OH and Z₂ is H.

33. The compound of claim 28 wherein Q₁ is —OH.

34. The compound of claim 33 wherein Q₂ is:

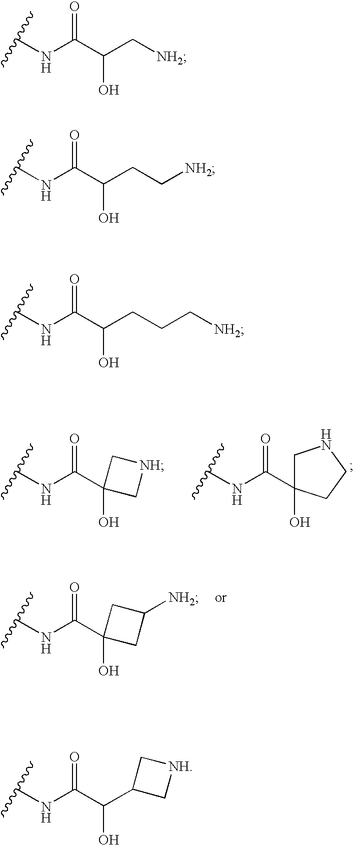

35. The compound of claim 34 wherein Z₁ is H and Z₂ is —OH.

36. The compound of claim 27 wherein Q₅ is —OH.

37. The compound of claim 36 wherein Q₁ is amino.

38. The compound of claim 37 wherein Q₂ is:

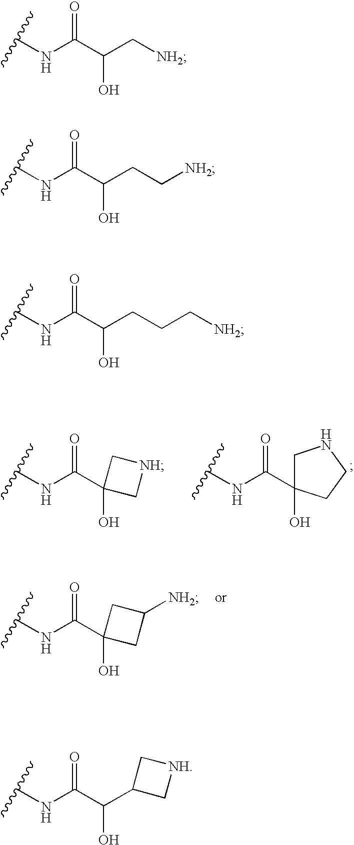

39. The compound of claim 38 wherein Z₁ is H and Z₂ is —OH.

40. The compound of claim 38 wherein Z₁ is —OH and Z₂ is H.

41. The compound of claim 36 wherein Q₁ is —OH.

42. The compound of claim 41 wherein Q₂ is:

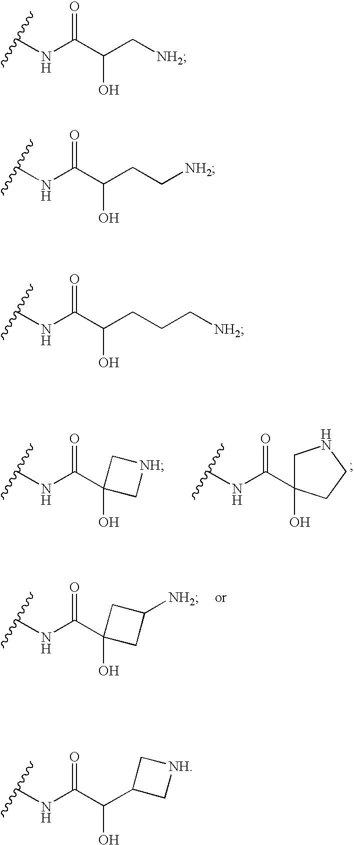

43. The compound of claim 42 wherein Z₁ is H and Z₂ is —OH.

44. The compound of claim 26 having the configuration:

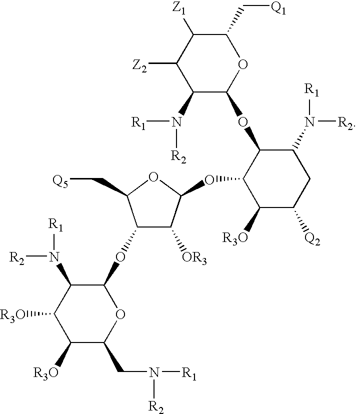

45. A pharmaceutical composition comprising a compound of claim 26, or a stereoisomer, pharmaceutically acceptable salt or prodrug thereof, and a pharmaceutically acceptable carrier, diluent or excipient.

46. A method of treating a bacterial infection in a mammal comprising administering to the mammal an effective amount of a compound of claim 26.

47. A compound having the following formula IV: 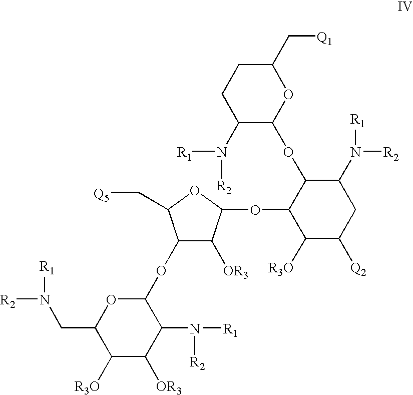
or a stereoisomer, prodrug or pharmaceutically acceptable salt thereof,
wherein:
Q₁ is —OH, a protected hydroxyl, amino or a protected amino group;
Q₂ is 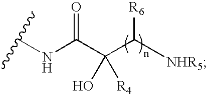
Q₅ is —OH, a protected hydroxyl, amino or a protected amino group;
each R₁ and R₂ is, independently, H or an amino protecting group;
each R₃ is, independently, H or a hydroxyl protecting group;
each R₄, R₅ and R₆ is, independently, H or C₁-C₆ alkyl, and R₄ and R₅ together with the atoms to which they are attached form a carbocyclic or heterocyclic ring having from 4 to 6 ring atoms, or R₅ and R₆ together with the atoms to which they are attached form a carbocyclic or heterocyclic ring having from 4 to 6 ring atoms, or R₄ and R₆ together with the atoms to which they are attached form a carbocyclic ring having from 4 to 6 ring atoms; and
n is an integer from 1 to 3.

48. The compound of claim 47 wherein each R₁, R₂ and R₃ are H.

49. The compound of claim 48 wherein Q₅ is amino.

50. The compound of claim 49 wherein Q₁ is amino.

51. The compound of claim 50 wherein Q₂ is:

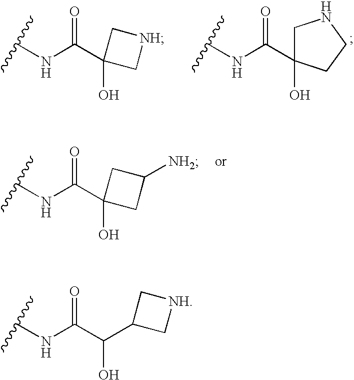

52. The compound of claim 49 wherein Q₁ is —OH.

53. The compound of claim 52 wherein Q₂ is:

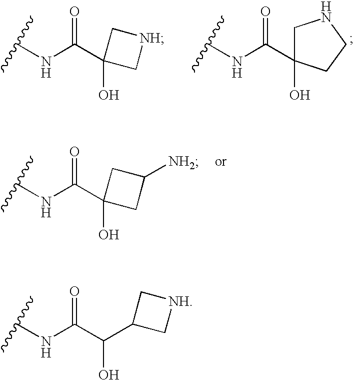

54. The compound of claim 48 wherein Q₅ is —OH.

55. The compound of claim 54 wherein Q₁ is amino.

56. The compound of claim 55 wherein Q₂ is:

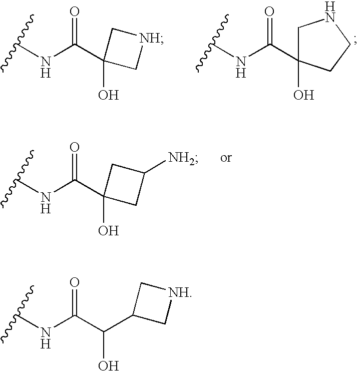

57. The compound of claim 54 wherein Q₁ is —OH.

58. The compound of claim 57 wherein Q₂ is:

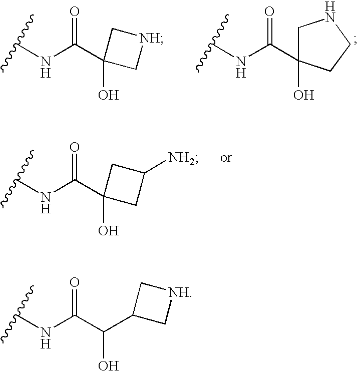

59. The compound of claim 47 having the configuration:

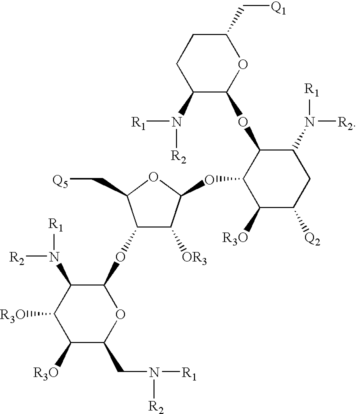

60. A pharmaceutical composition comprising a compound of claim 47, r a stereoisomer, pharmaceutically acceptable salt or prodrug thereof, and a pharmaceutically acceptable carrier, diluent or excipient.

61. A method of treating a bacterial infection in a mammal comprising administering to the mammal an effective amount of a compound of claim 47.
